(19) United States
(12) Patent Application Publication
(10) Pub. No.: US 2023/0365636 A1
(43) Pub. Date: Nov. 16, 2023

(54) BIOORTHOGONAL REPORTER GENE SYSTEM

(71) Applicant: Technische Universität München, Munchen (DE)

(72) Inventors: Volker MORATH, München (DE); Arne SKERRA, Dachau (DE); Wolfgang WEBER, München (DE); Katja FRITSCHLE, München (DE)

(21) Appl. No.: 18/029,516

(22) PCT Filed: Nov. 16, 2021
(86) PCT No.: PCT/EP2021/081775

§ 371 Date: Mar. 30, 2023

(30) Foreign Application Priority Data
Nov. 16, 2020 (EP) 20207716.0

(51) Int. Cl. C07K 14/47; C12N 15/86; G01N 33/566; C07K 14/73; C07K 14/705; A61K 51/08; A61K 51/04

(52) U.S. Cl. CPC C07K 14/47; C12N 15/86; G01N 33/566; C07K 14/70514; C07K 14/70521; A61K 51/08; A61K 51/0497; C12N 2750/14143; C07K 2319/035; C07K 2319/03; C07K 2319/60

(57) ABSTRACT

The present invention relates to a nucleic acid molecule encoding a fusion protein comprising (i) a secretory signal peptide; (ii) a lipocalin-derived binding protein specifically binding to an exogenous ligand; and (iii) a glycosylphosphatidylinositol (GPI) anchored and/or transmembrane domain.

### RELATED PATENT APPLICATIONS

This patent application is a 35 U.S.C. 371 national phase patent application of PCT/EP2021/081775 filed on Nov. 16, 2021, entitled “BIOORTHOGONAL REPORTER GENE SYSTEM”, naming Volker MORATH, Arne SKERRA, Wolfgang WEBER, and Katja FRITSCHLE as inventors, and designated by attorney docket no. AC1553 PCT, which claims priority to European Application No. 20207716.0 filed on Nov. 16, 2020, entitled “BIOORTHOGONAL REPORTER GENE SYSTEM,” naming Volker MORATH, Arne SKERRA, Wolfgang WEBER, and Katja FRITSCHLE as inventors, and designated by attorney docket no. AD1553 EP. The entire content of the foregoing patent applications is incorporated herein by reference, including all text, tables and drawings.

### SEQUENCE LISTING

The instant application contains a Sequence Listing which has been submitted electronically in ASCII format and is hereby incorporated by reference in its entirety. Said ASCII copy is named Sequence_Listing and is 36 kilobytes in size.

The present invention relates to a nucleic acid molecule encoding a fusion protein comprising (i) a secretory signal peptide; (ii) a lipocalin-derived binding protein that specifically binds to an exogenous ligand; and (iii) a glycosylphosphatidylinositol (GPI) anchored and/or transmembrane domain.

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The life sciences have greatly benefited from the development of reliable reporter gene systems such as fluorescent proteins, luciferases or enzymes. These systems enabled the time-resolved quantification of transcription, analysis of cis-acting genetic elements, understanding of developmental processes, determining protein localization, construction of synthetic biosensors and will in the future be applied in synthetic biology to monitor the dynamics of artificial biological systems (Ghim et al., 2010).

First steps towards the use of engineered cells in a medical context are exemplified by the clinical studies and recent FDA approval of chimeric antigen receptors (CARs) in T-cells, which harness the immune system to specifically combat cancer. Furthermore, therapeutic interventions by gene and cell therapy vectors such as Adenovirus- or AAV-based vectors have become established therapies today. Generally, there is desire to monitor or image such genetically modified cells in the body of the patient during and after therapy.

A reporter protein that can be used in various animal models as well as in human patients requires good tissue penetration of the resulting signal, which is unfortunately not the case for conventional, light-based reporter proteins. In contrast, radioactive imaging technologies such as Positron Emission Tomography (PET) and Single Photon Emission Computed Tomography (SPECT) are routinely applied in nuclear medicine and medical care and allow the highly sensitive, spatiotemporally resolved and quantitative monitoring of signals within patients.

In this context, there is an ongoing need for a reporter protein system suitable to reliably monitor these gene/cell therapies and/or engineered/transformed cells within a patient (Serganova and Blasberg, 2005, Serganova et al., 2008, Brader et al., 2013). This need is addressed by the provision of the embodiments characterized in the claims.

Accordingly, the present invention relates in a first aspect to a nucleic acid molecule encoding a fusion protein comprising (i) a secretory signal peptide; (ii) a lipocalin-derived binding protein that specifically binds to an exogenous ligand; and (iii) a glycosylphosphatidylinositol (GPI) anchored and/or transmembrane domain.

The term “nucleic acid molecule”, also referred to a nucleic acid sequence or polynucleotide herein, as used herein includes DNA, such as cDNA or genomic DNA, synthetic DNA and RNA. It is understood that the term “RNA” as used herein comprises all forms of RNA including mRNA. Both, single-strand as well as double-strand nucleic acid molecules are encompassed by this term. Further included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), peptide nucleic acid (PNA) and locked nucleic acid (LNA) (see (Braasch and Corey, 2001)).

The nucleic acid molecules of the invention can e.g. be synthesized by standard chemical synthesis methods or isolated from natural sources or produced semi-synthetically, i.e. by combining chemical synthesis and isolation from natural sources. Ligation of the coding sequences to transcriptional regulatory elements and/or to other amino acid encoding sequences can be carried out using established methods, such as restriction digest, polymerase chain reaction (PCR), ligation and molecular cloning.

The term “(poly)peptide” in accordance with the present invention describes a group of molecules which comprises the group of peptides, consisting of up to 50 amino acids, as well as the group of polypeptides, consisting of more than 50 amino acids. Also encompassed by the term “(poly)peptide” are proteins as well as fragments of proteins. (Poly)peptides may further form dimers, trimers and higher oligomers, i.e. consisting of more than one (poly)peptide molecule. (Poly)peptide molecules forming such dimers, trimers etc. may be identical or non-identical. The corresponding higher order structures are, consequently, termed homo- or heterodimers, homo- or heterotrimers etc. Homo- or heterodimers etc. also fall under the definition of the term “(poly)peptide”. The terms “polypeptide” and “protein” are used interchangeably herein and also refer to naturally modified polypeptides wherein the modification is effected e.g. by glycosylation, acetylation, phosphorylation and the like. Such modifications are well known in the art.

The “fusion protein” as used herein refers to a protein, wherein (i) a secretory signal peptide; (ii) a lipocalin-derived binding protein specifically binding to an exogenous ligand; and (iii) a glycosylphosphatidylinositol (GPI) anchored and/or transmembrane domain are fused to each other directly or via a peptide-linker. The term “peptide-linker”, as used in accordance with the present invention, preferably relates to peptide or polypeptide linkers, i.e. a sequence of amino acids.

A peptide-linker as envisaged by the present invention is at least 1 amino acid in length. Preferably, the peptide-linker is 1 to 100 amino acids in length. More preferably, the peptide-linker is 5 to 50 amino acids in length and even more preferably, the peptide-linker is 10 to 20 amino acids in length. Preferably, the peptide-linker is a flexible peptide-linker using e.g. the amino acids glycine and/or serine. Preferably, the peptide-linker sequences are (Gly₄Ser)₃, or (Gly₄Ser)₂. The length and sequence of a suitable peptide-linker depends on the composition of the respective specific fusion protein. Methods to test the suitability of different peptide-linkers are well known in the art and include e.g. the comparison of the binding affinity to the exogenous ligand or the protein stability or the production yield (surface density of the functional protein) of the fusion protein with and without peptide-linkers.

In the case of a “fusion protein”, conjugation may be carried out by recombinant DNA technology using well established techniques. As a result, the conjugate is created as one continuous polypeptide chain through the joining of two or more genes that originally code for separate molecules. Translation of this fusion gene results in a fusion protein with functional properties derived from each of the original molecules (i) to (iii). Suitable vectors for the recombinant production of the fusion protein are known in the art and will be described herein below.

The term “secretory signal peptide” designates a short peptide (e.g. 15-50 amino acids long) that can be found naturally at the N-terminus of the majority of newly synthesized proteins that are destined towards the secretory pathway. A typical secretory signal peptide comprises three distinct regions: a polar N-terminal end (n-region) that may have a net positive charge, a central hydrophobic core (h-region) that consists of 6-15 hydrophobic amino acids, and a polar C-terminal (c-region) end that contains amino acids with small side chains. Such a signal peptide that can also be found on the N-terminus of a type I membrane-bound protein. Type I membrane-bound proteins are anchored to the lipid membrane with a stop-transfer anchor sequence and have their N-terminal domains targeted to the endoplasmic reticulum (ER) lumen during biosynthesis and to the extracellular space if mature forms are located on the cell membrane as anticipated in this invention.

In some embodiments, the hydrophobic C-terminal sequence of the translocated protein may be cleaved and replaced by a glycosylphosphatidylinositol (GPI) anchor such that the protein finally remains attached to the outer leaflet of the cell membrane (Paulick and Bertozzi, 2008).

The term “that specifically binds to an exogenous ligand” relates to a molecule that is capable of specifically binding to (also referred to herein as “specifically interacting with”) an exogenous ligand but is not capable of or essentially not capable of cross-reacting with a different compound. Cross-reactivity of a panel of molecules under investigation may be tested, for example, by assessing binding of said panel of molecules to the exogenous ligand as well as to a number of more or less (structurally and/or functionally) closely related compounds. Only those molecules that bind to the exogenous ligand but do not or do not essentially bind to any of the other compounds are considered specific for the exogenous ligand.

The term “a molecule that essentially does not cross-react”, as used herein, refers to a molecule that binds to the exogenous ligand with at least 5-times higher affinity as compared to a different exogenous ligand of similar structure, more preferably at least 10-times higher affinity, such as e.g. at least 50-times higher affinity, more preferably at least 100-times higher affinity, such as e.g. at least 250-times higher affinity. Even more preferably, it binds with at least 500-times higher affinity to the exogenous ligand than to a different compound of similar structure and most preferably with at least 1000-times higher affinity.

The term “ligand” as used herein refers to the compound which is specifically bound by a lipocalin-derived binding protein according to the invention. As discussed, a naturally-occurring lipocalin can be engineered into a lipocalin-derived binding protein that is capable of specifically binding virtually any desired compound. The nature of the ligand is, thus, not particularly limited. The ligand can be, for example, a (poly)peptide, a carbohydrate or a small organic compound. As is evident from the appended examples, a lipocalin-derived binding protein according to the invention may, for example, specifically bind to Bn-CHX-A″-DTPA⋅Me, colchicine or a demethylated colchicine, petrobactin, dig(it)oxigenin or fluorescein. As will be further discussed herein below, the ligand according to the present invention is preferably linked to a radionuclide. Also, the ligands as used in the appended examples are linked to a radionuclide. A radionuclide is a label allowing the detection of the ligand, for example, in vivo within a subject.

The term “exogenous” as used in connection with the ligand indicates that the ligand is an exogenous factor that does no naturally occur in the cell wherein the fusion protein of the invention is to be incorporated and, if used for any medical or diagnostic application, also does not naturally occur in the subject to be treated or diagnosed. The subject is preferably human. Hence, an exogenous factor originates outside that cell and/or subject, as opposed to an endogenous factor.

The term “a lipocalin-derived binding protein specifically binding to an exogenous ligand” may be one or more than one lipocalin-derived binding protein and is preferably one lipocalin-derived binding protein. In the case of more than one lipocalin-derived binding protein each type of lipocalin-derived binding protein may specifically bind to a distinct exogenous ligand. Hence, in the case of a first and a second lipocalin-derived binding protein the first lipocalin-derived binding protein may specifically bind to a first exogenous ligand and the second lipocalin-derived binding protein specifically may bind to a second exogenous ligand. Alternatively, it is possible to use two or more copies of the same lipocalin-derived binding protein. In this case, more than one ligand can be bound to the fusion protein. This in turn can increase the signal strength or specificity, for instance, in case the ligand is bound to a label, which is preferably a radionuclide as will be described in more detail herein below.

Lipocalin-derived binding proteins, also referred to as Anticalins, represent a class of non-immunoglobulin binding proteins based on the human lipocalin scaffold. Lipocalins comprise a diverse family of small (20 kDa) extracellular proteins that occur in many species ranging from bacteria to humans and serve for the transport or scavenging of physiological compounds. Despite mutually low sequence homology, the three-dimensional fold of lipocalins is highly conserved (Schiefner and Skerra, 2015).

The single chain molecular architecture of lipocalin-derived binding proteins is dominated by a compact eight-stranded anti-parallel β-barrel. At the open end of the barrel there are four loops connecting each pair of β-strands. The four structurally variable loops are referred to herein as “loop regions”, whereas the remainder of the protein makes up the framework or “frame regions”. Thus, similar to the structure of antibodies, the lipocalin-derived binding proteins according to the present invention are essentially made of conserved frame(work) regions that are generally not directly involved in the binding to the exogenous ligand, as well as hypervariable, specificity-determining segments with amino acid residues being involved in the binding to the exogenous ligand (here the loop regions, which might be seen as resembling the CDRs in antibodies).

Hence, lipocalin and also lipocalin-derived binding proteins consist of frame regions and loop regions according to the following scheme:
Frame 1—Loop 1—Frame 2—Loop 2—Frame 3—Loop 3—Frame 4—Loop 4—Frame 5

The lipocalin-derived binding protein is preferably a lipocalin 2 (Lcn2)-derived binding protein. Lipocalin-2 (Lcn2), also known as neutrophil gelatinase-associated lipocalin (NGAL), is a protein that in humans is encoded by the LCN2 gene. Human LCN2 mRNA is, for example, represented by the NCBI Reference Sequence: NM_005564.5 (as available on Mar. 12, 2019) and the amino acid sequence of human Lcn2 protein including the signal peptide is, for example, represented by the UniProt ID P80188-2 (as available on Nov. 1, 1995).

The lipocalin-derived binding protein binds the exogenous ligand with increased preference with a K_{D} of 200 nM or lower, 100 nM or lower, 50 nM or lower, 10 nM or lower, 1 nM and 500 pM or lower.

The term “K_{D}” refers to the equilibrium dissociation constant (the reciprocal of the equilibrium binding constant) and is used herein according to the definitions provided in the art.

The K_{D} value with which the lipocalin-derived binding protein binds the exogenous ligand can be determined by well known methods including, without being limiting, fluorescence titration, competition ELISA, calorimetric methods, such as isothermal titration calorimetry (ITC), flow cytometric titration analysis (FACS titration), radioligand binding assays and surface plasmon resonance spectroscopy (BIAcore). Such methods are well known in the art and have been described e.g. in De Jong, L. A. A. et al. [2005] J. Chromatogr. B 829(1-2):1-25; Heinrich, L. et al. [2010] J. Immunol. Methods 352(1-2):13-22.

Preferably, ELISA or competition ELISA or surface plasmon resonance (BIAcore) is employed to ensure that the K_{D} of the lipocalin-derived binding protein binds the exogenous ligand with the required K_{D}. Even more preferably, the K_{D} is determined by surface plasmon resonance (BIAcore).

A “glycosylphosphatidylinositol (GPI) anchored domain” as used herein relates to a domain comprising a GPI anchor. GPI is composed of a phosphatidylinositol group linked through a carbohydrate-containing linker (glucosamine and mannose glycosidically bound to the inositol residue) and via an ethanolamine phosphate (EtNP) bridge to the C-terminal amino acid of a mature protein. The two fatty acids within the hydrophobic phosphatidyl-inositol group anchor the protein to the cell membrane.

The term “transmembrane domain” as used herein designates any cell membrane-spanning protein domain. Transmembrane domains (TMDs) generally consist predominantly of nonpolar amino acid residues and may traverse the cell membrane bilayer once or several times. The transmembrane domain is preferably a single-pass transmembrane domain. TMDs usually consist of α-helices. The peptide bond is intrinsically polar and can form internal hydrogen bonds between carbonyl oxygens and amide nitrogens. Within the lipid bilayer, where water is essentially excluded, peptides usually adopt the α-helical configuration that maximizes their internal hydrogen bonding. A helix length of 18-21 amino acid residues is sufficient to span the usual width of a lipid bilayer.

It is, thus, to be understood that the fusion protein being encoded by the nucleic acid molecule comprises (i) a secretory signal peptide; (ii) a lipocalin-derived binding protein specifically binding to an exogenous ligand; and (iii) a glycosylphosphatidylinositol (GPI) anchored and/or transmembrane domain in that particular order (i) to (iii) from the N-terminus to the C-terminus of the fusion protein or, alternatively, from the C-terminus to the N-terminus, in this case without a secretory signal peptide, depending on the type of transmembrane domain to be used. The orientation has to be selected—based on the type of transmembrane domain to be used—such that after the incorporation of the transmembrane domain into the cell membrane a lipocalin-derived binding protein specifically binding to an exogenous ligand is found on the extracellular side.

It is evident from the examples herein below that once the nucleic acid molecule of the invention is expressed in a cell the encoded fusion protein of the invention is processed into the mature fusion protein (lacking the signal peptide (i)) and incorporated into the plasma membrane of the cell via the transmembrane domain or a glycosylphosphatidylinositol (GPI) anchor, so that the lipocalin-derived binding protein specifically binding to an exogenous ligand is anchored and present on the extracellular surface of the cell. Once the exogenous ligand comes into contact with a cognate lipocalin-derived binding protein the lipocalin-derived binding protein and the exogenous ligand firmly bind to each other. This binding can be detected (e.g. visualized) by labelling the exogenous ligand with a detectable label as will be described in greater detail herein below.

The anchored lipocalin-derived binding protein encoded by a nucleic acid molecule of the invention and the exogenous ligand preferentially form a reporter system. These compounds making up the reporter system can be administered to a patient or subject (preferably a human patient or subject) either simultaneously or sequentially (wherein in the latter case the nucleic acid molecule of the invention is generally to be administered first).

The exogenous ligand is preferably administered systemically, e.g. via intravenous transfusion or injection. Upon administration the exogenous ligand is distributed among the blood compartment and is selectively bound by the lipocalin-derived binding protein. This in turn allows the monitoring of the cells, e.g. with a living organism with minimal functional impairment and, on the other hand, with a minimal background signal.

As discussed, the lipocalin-derived binding proteins as used herein are also also-known as Anticalins. Anticalins constitute an emerging class of artificial binding proteins obtained by combinatorial design based on the compact and robust human lipocalin scaffold. Due to their human origin, the lipocalin-derived binding proteins according to the present invention have low immunogenic potential, and in several clinical trials Anticalins with different target specificities have demonstrated safety. Moreover, these lipocalin-derived binding proteins according to the present invention show remarkable target specificity, with dissociation constants in the nanomolar and even picomolar range (Table 1). The lipocalin-derived binding protein is preferably selected from SEQ ID NOs 3/4 and 5/6, noting that the lipocalin-derived binding protein of SEQ ID NO: 3/4 is capable of specifically binding DTPA and the lipocalin-derived binding protein of SEQ ID NO: 5/6 is capable of specifically binding colchicine.

As will be further detailed herein below, the Anticalin-based reporter system as provided herein is in particular suitable for therapeutic and diagnostic purposes. The Anticalin-based reporter system is particularly well suited for applications in CAR T-cell anti-cancer therapy, gene therapy and diagnostics.

An antibody-based reporter system which is similar to the Anticalin-based reporter system as provided herein is described in (Krebs et al., 2018). To design a reporter system based on an Anticalin instead of an antibody is technically advantageous because an Anticalin by nature is a single polypeptide chain molecule whereas an antibody comprises heavy and light chains. The less complicated and less interference-sensitive folding process of the Anticalin as compared to an antibody or antibody fragment ensures efficient folding within the secretory pathway and, thus, allows for high expression on the cell surface. Especially when used to genetically tag CAR T-cells, the use of an immunoglobulin folding motif with light and heavy chain may prove disadvantageous as the light chains of i) the antibody-based reporter gene and ii) the chimeric antigen receptor (CAR) may mutually interfere with correct folding. While based on an antibody, a single-chain fragment (scFv) may be designed as was done by (Krebs et al., 2018),

Anticalins display an improved binding affinity to their cognate exogenous ligands as compared to a single-chain fragment of an antibody. (Krebs et al., 2018) designed an antibody-based reporter system wherein the binding between the single-chain fragment and the exogenous ligand is irreversible (i.e. infinite). Due to the superior binding affinity of an Anticalin the Anticalin-based reporter system as provided herein is stable in vivo without the need of an irreversible covalent bond between the Anticalin and the cognate exogenous ligand. Furthermore, the antibody used in (Krebs et al., 2018) was of murine origin and only of limited value for clinical translation. As can be taken from the examples, the system according to the invention allows the clinical monitoring of emerging therapies such as CAR T-cells or gene therapy. The reversible binding between the Anticalin and the cognate exogenous ligand makes the Anticalins-based reporter system as provided herein more flexible and easier to use for such applications.

In summary, the disadvantages of antibody-based reporter system and in particular the scFv fragment-based reporter system of Krebs et al., 2018 are 1) the tendency of the scFv fragment to form oligomers (e.g. Hudson & Kortt, 1999), 2) low surface expression of the system, 3) the murine origin of the system and the resulting potential immunogenicity in humans, and 4) the cross-reactivity of the radioligand (lanthanoid (S)-2-(4-acrylamidobenzyl)-DOTA (AABD)). As an acrylamide derivative the radioligand may unspecifically bind to structures other than the DOTA antibody reporter 1 (DAbR1) reporter protein. On the other hand, the advantages of the lipocalin-based reporter proteins according to the invention include, but are not limited to: 1) the human origin of the protein scaffold, 2) the smaller size of the reporter protein, 3) the single-chain protein architecture, 4) the high in vivo stability of the folded protein and 5) the absence of oligomerisation tendency.

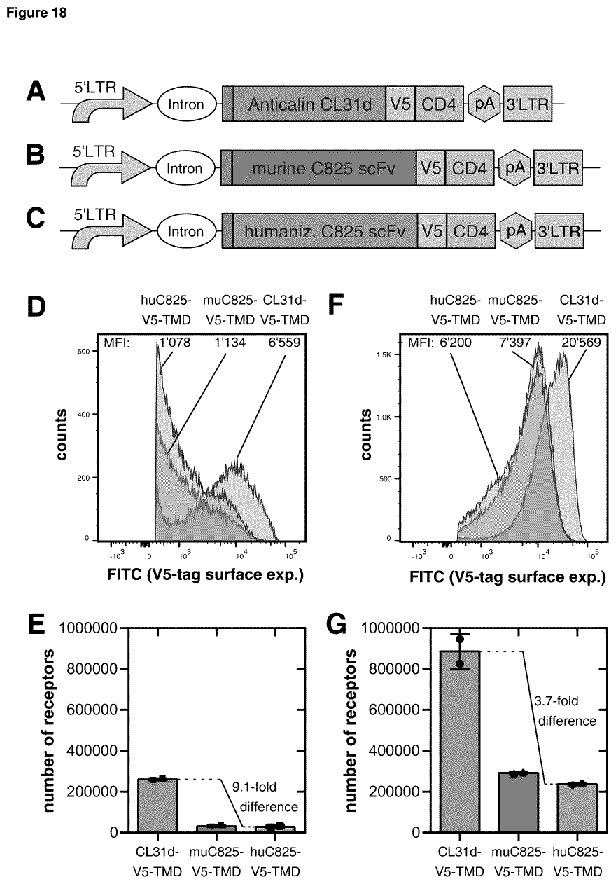

In addition, Example 7 and FIG. 18 provide a side-by-side comparison of Anticalin-based reporter proteins according to this invention with an scFv fragment (C825) specific for DOTA:metal complexes known in the art (Dacek et al., (2021)) (EP 3256164 B1, WO 2019/060750A2) similar to the one used before for an scFv-based reporter system (Krebs et al., 2018). It is demonstrated that the Anticalin-based reporter proteins display an about 9-fold higher expression level on the cell surface as compared to the prior art, with an scFv fragment displayed in the same setting, including the V5-epitope tag and the same membrane anchor. The 9-fold higher expression level on the cell surface was totally unexpected and leads to technical advantages. This is because the 9-fold higher expression level on the cell surface will lead to the binding of a significantly larger number of radioligands (presumably also about 9-fold larger) to the surface of each cell. This will in turn result in a significantly more sensitive detection of transduced or transfected cells in PET imaging studies.

Thus, the Anticalin-based reporter system provided herein is in several aspects superior to antibody or antibody fragment-based reporter systems, including the treatment and diagnosis of tumors, and in particular for the in vivo imaging of diseases and therapies.

Means and methods for introducing and expressing the nucleic acid molecule of the invention in a cell, such that the encoded fusion protein is anchored in the cell membrane, will be discussed herein below.

In accordance with a preferred embodiment of the first aspect of the invention the exogenous ligand is linked to a radionuclide, wherein the radionuclide is preferably selected from C-11, F-18, Sc-44, Sc-47, Cu-64, Ga-68, Y-86, Y-90, Zr-89, Tc-99m, In-111, I-123, I-124, I-131, Tb-152 and Lu-177, Bi-213, Ac-225.

The radionuclide may be directly or indirectly linked (i.e. via linker) to the exogenous ligand. The linker is preferably an organic compound. Also, the linker preferably links the exogenous ligand and the radionuclide via 5 to 30 connecting atoms. As illustrated in the examples the linker is most preferably a polyethylene glycol (PEG)-linker.

As is illustrated in the appended examples, the radiolabeling of the exogenous ligand allows for detecting the exogenous ligand, e.g. within a living subject, and also allows for the detection and quantification of cells carrying the fusion protein of the invention in the cell membrane, wherein these cell have bound the exogenous ligand. Each cell comprises a plurality of fusion proteins within its cell membrane, such that also a plurality of the exogenous ligand is bound to the cell. Thereby labelled exogenous ligands come into close proximity on the cell surface which can be distinguished from unbound, free exogenous ligands, which are rapidly excreted via the kidneys or the hepato-biliary route. For this diagnostic application the exogenous ligand needs to be administered to a subject comprising such cells. As a consequence, the location of the cells, transfected with the nucleic acid according to this invention and having bound the ligand, can be visualized and quantified in the subject.

A radionuclide may be a diagnostic radionuclide or a therapeutic radionuclide and is preferably a diagnostic radionuclide. A therapeutic radionuclide is a radionuclide that can be used for the treatment or prevention of a disease. For instance, a therapeutic radionuclide may be used to specifically kill cells that accumulate the radionuclide. The radionuclides being best suited for therapy are generally those emitting high energy ionizing radiation with short penetration into the tissue, e.g. α (alpha) or β⁻ (beta) emitters. This ionizing radiation causes damage to the DNA and, thus, harm the cells. On the other hand, the radiation emitted by diagnostic radionuclides (e.g. β⁺ (positron) radiation for PET or γ (gamma) radiation for SPECT) essentially does not kill cells. Diagnostic radionuclides used, for example, for imaging procedures are generally radionuclides emitting γ (gamma) radiation, directly or after positron annihilation. γ (gamma) radiation is able to penetrate tissue.

The following lists of radionuclides are non-limiting but preferred examples of radionuclides that are used in the art for diagnostic (C-11, F-18, Sc-44, Cu-64, Ga-68, Y-86, Zr-89, Tc-99m, In-111, I-123, I-124, Tb-152, Tb-155) or therapeutic (Sc-47, Y-90, 1-131, Tb-149, Tb-161, Lu-177, Bi-213, Ac-225) purposes.

While a (1) diagnostic radionuclide such as Fluorine-18 is the preferred label it is also possible to link the Anticalin-bound exogenous ligand moiety to (2) (super)paramagnetic (nano)beads for Magnetic resonance imaging (MRI) detection or magnetic-activated cell sorting (MACS), (3) pharmaceutically active substances, such as growth factors, toxins or immunomodulators, or (4) therapeutic radionuclides to harm the cells.

In accordance with another preferred embodiment of the first aspect of the invention the encoded fusion protein further comprises a peptide affinity tag, preferably a V5, Strep-tag II, Flag, myc, HA, Spot, T7 or NE tag.

Affinity tags are peptide sequences genetically incorporated into the fusion protein of the invention, preferentially at the outer side of the cell membrane. This affinity tag can be used together with a cognate antibody or another binding protein, e.g. streptavidin, for the detection of the invention by various methods such as flow cytometry, immunofluorescence microscopy, western blot detection or immunohistochemistry (IHC). Furthermore, the affinity tag can be used together with a cognate antibody, or streptavidin/Strep-Tactin, and/or superparamagnetic microbeads for the isolation of cells labelled with the invention via magnetic-activated cell sorting (MACS) (Miltenyi and Schmitz, 2000, Grutzkau and Radbruch, 2010)

In the examples herein below the V5-epitope tag is used as a peptide affinity tag, so that the peptide affinity tag is most preferably a V5-epitope tag (Southern et al., 1991, Dunn et al., 1999).

The affinity tag generally is located on the extracellular part of the fusion protein of the invention. It is preferably inserted between (ii) the lipocalin-derived binding protein specifically binding to an exogenous ligand; and (iii) a glycosylphosphatidylinositol (GPI) anchored and/or transmembrane domain. The affinity tag is most preferably inserted at this position in the form of the linker of SEQ ID NO: 7/8, which linker comprises a V5-epitope tag. This has the advantage that the tag is available on the outside of a cell but very likely does not interfere with the ability of a lipocalin-derived binding protein to specifically bind its exogenous ligand.

In accordance with a further preferred embodiment of the first aspect of the invention the encoded fusion protein further comprises a fluorescent protein, preferably a monomeric autofluorescent protein such as mRuby3, GFP, eGFP, sfGFP, UnaG, miRFP703 or miRFP720.

The fluorescent protein is used to enable a visual or spectrophotometric readout on the fusion protein of the invention, on a cellular and sub-cellular level.

GFP (green fluorescent protein) and its variants (e.g. eGFP and sfGFP) are the most commonly used fluorescence tags. More advanced applications of GFP include using it as a folding reporter (fluorescent if folded, colorless if not).

mRuby3 (Bajar et al., 2016) is a monomeric bright red fluorescent protein derived from Entacmaea quadricolor which is has a low acid sensitivity.

UnaG is a basic (constitutively fluorescent) green fluorescent protein derived from Anguilla japonica. It has low acid sensitivity. It requires the cofactor bilirubin for fluorescence (Kumagai et al., 2013).

MiRFP703 (Shcherbakova et al., 2016) or miRFP720 (Shcherbakova et al., 2018) are monomeric near-infrared (NIR) fluorescent proteins that also require the cofactor bilirubin for fluorescence. Due to the low absorbance of different tissues for light of this wavelength and the low autofluorescence, these fluorescent proteins can be used for in vivo imaging experiments.

In the examples herein below mRuby3 and miRFP720 are used as the fluorescent protein, so that the fluorescent protein is most preferably the mRuby3 of SEQ ID NO: 17/18 or the miRFP720 of SEQ ID NO: 19/20.

The fluorescent protein preferably is located on the intracellular part of the fusion protein of the invention, such that it may not interfere with the ability of the lipocalin-derived binding protein to specifically bind its exogenous ligand.

The fluorescent protein is preferably separated from the reporter protein using a 2A self-cleaving peptide sequence. 2A self-cleaving peptide sequences (or 2A peptides) are a class of 18-22 residue peptides, which can induce ribosomal skipping during translation of a protein in a cell (see, for example, Liu et al. (2017)), Sci Rep., 7:2193).

The exemplified fusion protein of the invention without a fluorescent protein but comprising an anti-colchicine Anticalin, a linker with a V5-tag SEQ ID NO: 7/8 and the transmembrane domain of CD4 is shown in SEQ ID NO: 9/10. Said exemplified fusion protein with a mRuby3 label is shown in SEQ ID NO: 21/22 and said exemplified fusion protein with a miRFP720 label is shown in SEQ ID NO: 27/28. The fusion proteins comprising any one of SEQ ID NOs 21/22 and 27/28 are particularly preferred examples of the fusion protein of the invention. These SEQ ID NOs comprise the fluorescent protein within the intracellular part of the fusion protein.

The detection of the fluorescent protein can be used to detect the presence and location of the encoded fusion protein, for example in vivo. In case the fusion protein sits in a cell and that cell is to be administered to a subject and presence, location and movement of the cells in the subject can be detected. In case the exogenous ligand comprises a radionuclide as will be discussed herein below, the co-localization of the radionuclide and the fluorescent protein may indicate the binding of a lipocalin-derived binding protein of the fusion protein to the exogenous ligand.

In accordance with a preferred embodiment of the first aspect of the invention the exogenous ligand comprises a small molecule, wherein the small molecule is preferably selected from (i) a chelator, preferably a metal chelator complex, more preferably [(R)-2-amino-3-(4-aminophenyl)propyl]-trans-(S,S)-cyclohexane-1,2-diaminepentaacetic acid (p-NH₂-Bn-CHX-A″-DTPA), (ii) an alkaloid, preferably colchicine or a demethylated variant thereof, (iii) an iron-chelating siderophore, preferably petrobactin, enterobactin or bacillibactin, (iv) a plant steroid, preferably digoxigenin or digitoxigenin, and (v) an organic dye, preferably fluorescein or aminofluorescein, or the exogenous ligand is a peptide with at least 2 and less than 10 amino acid residues, preferably VFFAED (SEQ ID NO: 29).

It is to be understood that in accordance with a preferred embodiment the lipocalin-derived binding protein specifically binds a small molecule. Hence, the small molecule comprises an “epitope” which is recognized and specifically bound by the lipocalin-derived binding protein.

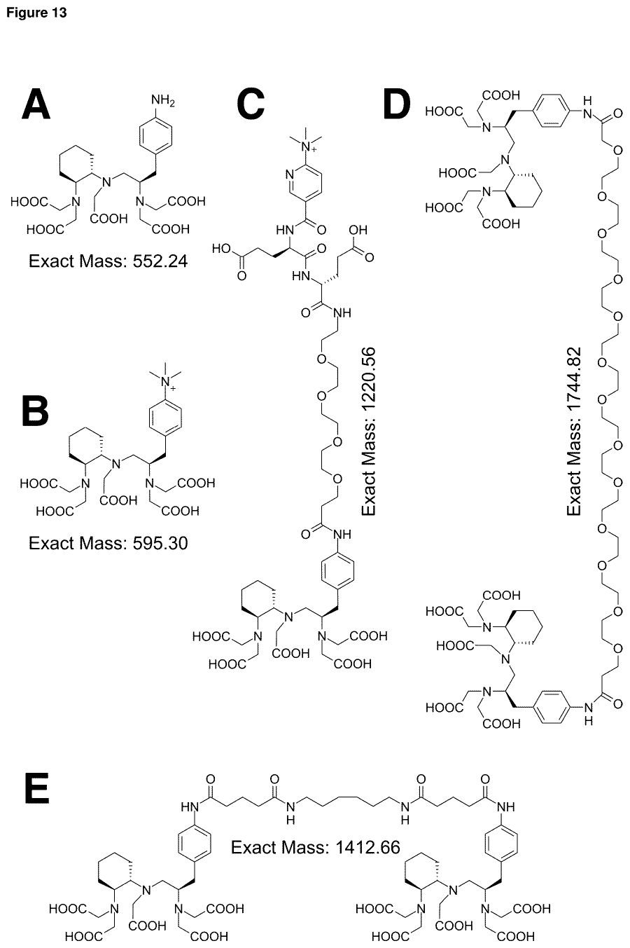

A small molecule is a low molecular weight compound which is by definition not a polymer. The upper molecular weight limit for a small molecule is preferably 2000 Da, more preferably 1500 Da and most preferably 1200 Da. Libraries of small organic molecules and high-throughput techniques for screening such libraries with a binding protein, such as an antibody or an Anticalin, are established in the art. The small molecule is preferably an organic small molecule. Particularly, preferred examples of small molecules comprised by the exogenous ligand are shown in FIG. 13A to E. Among these small molecules the small molecule as shown in FIG. 13C is most preferred.

The present invention is also directed to an exogenous ligand comprising a small molecule selected from the small molecules as shown in FIG. 13A to E as well as a small molecule selected from the small molecules as shown in FIG. 13A to E. Again, the small molecule as shown in FIG. 13C is most preferred.

Chelators are small molecules with two or more separate interacting groups that can bind to a single central metal ion, forming a chelator complex. The chelator p-NH₂-Bn-CHX-A″-DTPA is used in the art together with a radionuclide (e.g. Y-90) as a therapeutic radiometal (Price and Orvig, 2014, Price et al., 2016).

Alkaloids are a class of nitrogenous organic compounds of plant origin which have pronounced physiological actions on humans. They include many drugs (morphine, quinine) and poisons (atropine, strychnine). The alkaloid colchicine is a drug that is used mainly in gout to treat an acute attack or to prevent an attack while starting uric acid-lowering therapy.

Iron-chelating siderophores are small, high-affinity iron-chelating compounds that are secreted by microorganisms such as bacteria and fungi and serve primarily to transport iron across cell membranes, although a broader range of siderophore functions is now being appreciated. Siderophores are among the strongest soluble Fe³⁺ binding agents known. Non-limiting but preferred examples of iron-chelating siderophores are petrobactin, enterobactin and bacillibactin.

Organic dyes possess a colour because they 1) absorb light in the visible spectrum (400-700 nm) or the near-infrared spectrum (700-950 nm), 2) have at least one chromophore (colour-bearing group), 3) have a conjugated system, i.e. a structure with alternating double and single bonds, and 4) exhibit resonance of electrons. Non-limiting but preferred examples of organic dyes are fluorescein and aminofluorescein. Fluorescein is commonly used in microscopy, in forensics and serology to detect latent blood stains, and in dye tracing. Fluorescein has an absorption maximum at 494 nm and also an emission maximum of 512 nm (in water). The excitation wavelength of aminofluorescein (5-AF) is 490 nm and the emission wavelength is 515 nm.

Peptides have been defined herein above. The peptide VFFAED (SEQ ID NO: 29) is a fragment of the Alzheimer amyloid peptide.

In accordance with a further preferred embodiment of the first aspect of the invention the secretory signal peptide is the signal peptide of a lipocalin, preferably of lipocalin 2 (Lcn2).

As discussed above, the binding proteins according to the invention are lipocalin-derived and in particular derivatives of Lcn2. For this reason, it is preferred to use as the secretory signal peptide the naturally occurring signal peptide of a lipocalin, preferably of Lcn2. In the examples herein below the natural signal peptide of lipocalin 2 (Lcn2) is used, which is shown in SEQ ID NO: 1/2. The secretory signal peptide therefore most preferably comprises or consists of SEQ ID NO: 1/2.

In accordance with a still further preferred embodiment of the first aspect of the invention the transmembrane domain is the transmembrane domain of CD4 or CD28 or a sequence stimulating the attachment of a GPI-anchor.

The transmembrane domains of CD4 or CD28 are single-span a-helical transmembrane domains. Moreover, CD4 and CD28 are both expressed on T-cells and can be found in the cell membrane of T-cells. The choice of a protein sequence that is already expressed in the desired cell type (like CD4 or CD8 TMD in T-cells) is expected to decrease the risk of unintended immunogenicity caused by the membrane protein of the invention. As will be further discussed herein below, the fusion protein being encoded by the first aspect to is to be preferably incorporated into the cell membrane of T-cells. The transmembrane domains of CD4 or CD28 preferably have the sequences of SEQ ID NO: 9/10 and 11/12, respectively.

Alternatively, a sequence that stimulates the attachment of a glycosylphosphatidylinisotol (GPI) membrane anchor to the fusion protein of the invention may be used. To this end, the last 1 to 20 amino acid residues of this protein together with the downstream propeptide sequence of a human protein that is naturally anchored to the cell membrane via a GPI-anchor may be fused to the lipocalin-derived binding protein or the V5-tag. Sequences may be derived from every GPI-anchored protein, preferentially from human Complement decay-accelerating factor (CD55; UniProtKB-P08174 (DAF_HUMAN) as available on Jul. 31, 2020) SEQ ID NO: 13/14 or CD59 glycoprotein (UniProtKB-P13987 (CD59_HUMAN) as available on Jul. 31, 2020) SEQ ID NO: 15/16.

The present invention relates in a second aspect a vector comprising the nucleic acid molecule of the first aspect, wherein the vector is preferably a retroviral vector, an adenoviral vector or adeno-associated vector (AAV).

The definitions and preferred embodiments of the first aspect of the invention apply mutatis mutandis to the second aspect of the invention.

The vector is typically an expression vector and can be a linear nucleic acid, plasmid, cosmid, virus, bacteriophage or another vector used conventionally e.g. in genetic engineering.

Generally, vectors can contain one or more origins of replication (ori) and inheritance systems for cloning or expression, one or more markers for selection in the host, e.g., antibiotic resistance, and one or more expression cassettes. In addition, the coding sequences cloned on the vector can be ligated to transcriptional regulatory elements and/or to other coding sequences using established methods. Such regulatory sequences are well known to those skilled in the art and include, without being limiting, regulatory sequences ensuring the initiation of transcription, internal ribosomal entry sites (IRES) (Owens et al., 2001), and, optionally, regulatory elements ensuring termination of transcription and stabilization of the transcript. Non-limiting examples for such regulatory elements ensuring the initiation of transcription comprise promoters, a translation initiation codon, enhancers, insulators and/or regulatory elements ensuring transcription termination. Further examples include Kozak sequences and intervening sequences flanked by donor and acceptor sites for RNA splicing. The vectors may also contain an additional expressible polynucleotide coding for one or more chaperones to facilitate correct protein folding. Vectors can also contain a sequence encoding a 2A self-cleaving peptide or 2A peptide (Kim et al., 2011). These peptides are a class of 18-22 amino acids long peptides, which can induce cleavage of a recombinant protein in a cell. This can be used to express a functional protein such as a chimeric antigen receptor (CAR) together with the membrane protein of the embodiment under the control of a single promoter.

Vector elements that have been optimized for the expression of Anticalins in bacteria have been described in the art, e.g. in (Gebauer and Skerra, 2012) and include the tetracycline promoter/operator (tet^{p/o}), which is chemically inducible with anhydrotetracycline, an affinity tag, such as e.g. Strep-tag II or the A3C5 tag, the rho-independent Ipp terminator as well as an ampicillin-resistance gene (β-lactamase), a truncated ColEI origin of replication, and, optionally, the intergenic region of the filamentous phage f1 for the biosynthesis of phagemid particles upon co-infection of E. coli with a helper phage.

Additional examples of suitable origins of replication include, for example, the full length ColE1, the SV40 viral and the M13 origins of replication, while additional examples of suitable promoters include, without being limiting, the cytomegalovirus (CMV) promoter, SV40-promoter, RSV-promoter (Rous sarcome virus), the lacZ promoter, EF-1α promoter, chicken β-actin promoter, CAG-promoter (a combination of chicken β-actin promoter and cytomegalovirus immediate-early enhancer), the gai10 promoter, human elongation factor 1α-promoter, AOX1 promoter, GAL1 promoter CaM-kinase promoter, the lac, trp or tac promoter, the T7 or T5 promoter, the lacUV5 or ara promoter, the Autographa califomica multiple nuclear polyhedrosis virus (AcMNPV) polyhedral promoter or a globin intron in mammalian and other animal cells. One example of an enhancer is e.g. the SV40-enhancer or the Woodchuck Hepatitis Virus (WHP) Posttranscriptional Regulatory Element (WPRE). Non-limiting additional examples for regulatory elements ensuring transcription termination include the SV40-poly-A site, the tk-poly-A site or the AcMNPV polyhedral polyadenylation signals. Further non-limiting examples of selectable markers include dhfr, gpt, neomycin, hygromycin, blasticidin or geneticin.

Preferably, the vector of the present invention is an expression vector. An expression vector according to this invention is capable of directing the expression of the nucleic acid molecule of the invention and, accordingly, of the fusion protein of the present invention encoded thereby.

The nucleic acid molecules and/or vectors of the invention as described herein above may be designed for introduction into cells by e.g. non-chemical methods (electroporation, sonoporation, optical transfection, gene electrotransfer, hydrodynamic delivery or naturally occurring transformation upon contacting cells with the nucleic acid molecule of the invention), chemical-based methods (calcium phosphate, liposomes, DEAE-dextrane, polyethylenimine, nucleofection), particle-based methods (gene gun, magnetofection, impalefection) phage vector-based methods and viral methods including infection. For example, expression vectors derived from viruses such as retroviruses, vaccinia virus, adeno-associated virus, adeno virus, herpes viruses, Semliki Forest Virus or bovine papilloma virus, may be used for delivery of the nucleic acid molecules into targeted cell population. Additionally, baculoviral systems can also be used as vectors in a eukaryotic expression system for the nucleic acid molecules of the invention.

The vector is preferably a retroviral vector, an adenoviral vector or adeno-associated vector (AAV) because these vectors are commonly used in gene therapy (Lundstrom, 2018). The most applied viral vectors are adenoviruses or viruses being based on adenoviruses. Naked dsDNA adenoviruses possess a packaging capacity of 7.5 kb of foreign DNA, providing short-term episomal expression of the gene of interest in a relatively broad range of host cells. The original adenovirus vectors generated strong immune responses, whereas the so-called gutted second and third generation vectors containing deletions have proven to elicit substantially reduced immunogenicity.

AAV vectors carry a small ssRNA genome, which allows packaging of only 4 kb inserts. Generally, AAV vectors are considered to generate low pathogenicity and toxicity and provides long-term episomal expression of the gene of interest. One limitation of using AAV relates to the immune response triggered by repeated administration and the seroprevalence against some serotypes. This problem has been addressed by applying a different AAV serotype for each re-administration. Another issue relates to the limited packaging capacity of foreign DNA into recombinant AAV particles. This shortcoming has been addressed by engineering dual AAV vectors.

Retroviruses possess a ssRNA genome with an envelope structure. Typically, retroviruses are randomly integrated into the host genome, resulting in transduced cells with a certain range of transgene expression strength. However, this shortcoming has triggered the development of safer vectors showing targeted integration and also of improved helper cell lines. Retroviruses can accommodate up to 8 kb of foreign inserts and have been considered the gold standard vectors for long-term gene therapy applications. One drawback of retroviruses is their incapability to infect non-dividing cells.

Retroviral vector system are known in the art and, for example reviewed in (Vargas et al., 2016). Also, adenoviral vector and adeno-associated vector (AAV) are known in the art; for review see, for example (Li and Samulski, 2020),

The present invention relates in a third aspect to a fusion protein being encoded by the nucleic acid molecule of the first aspect of the invention.

The definitions and preferred embodiments of the above aspects of the invention apply mutatis mutandis to the third aspect of the invention.

As discussed herein above, the fusion protein comprises (i) a secretory signal peptide; (ii) a lipocalin-derived binding protein specifically binding to an exogenous ligand; and (iii) a glycosylphosphatidylinositol (GPI) anchored and/or transmembrane domain. As also discussed herein above, said signal peptide ensures that the fusion protein is sorted towards the secretory pathway.

After protein translocation of the ER membrane, a secretory signal peptide is cleaved off. The fusion protein of the invention is thereby processed to its mature form and, after protein sorting, incorporated into the plasma membrane of a cell via its transmembrane domain or glycosylphosphatidylinositol (GPI) anchor.

Hence, the present invention also discloses a fusion protein being encoded by the nucleic acid molecule of the first aspect of the invention which only comprises the (ii) a lipocalin-derived binding protein specifically binding to an exogenous ligand and (iii) a transmembrane domain or glycosylphosphatidylinositol (GPI) anchor. Once said mature fusion protein is located in the plasma membrane of a cell, a lipocalin-derived binding protein specifically binding an exogenous ligand can be found on the outside of the cell (i.e. on the extracellular side).

The present invention relates in a fourth aspect to a cell being transduced or transfected with (or comprising) the nucleic acid molecule of the first aspect or the vector of the second aspect of the invention and/or comprising the fusion protein of the third aspect of the invention.

The definitions and preferred embodiments of the above aspects of the invention apply mutatis mutandis to the fourth aspect of the invention.

The cell is also referred to as a host cell. The host cell is preferably a human host cell. A host cell is preferably an ex vivo cell; i.e. a cell outside of a multi-cellular organism. It will be appreciated that the term “host cell or a non-human host transformed or transduced with the vector”, in accordance with the present invention, relates to a host cell or a non-human host that comprises the vector of invention.

Suitable prokaryotic hosts comprise e.g. bacteria of the species Escherichia, Corynebacterium (glutamicum), Pseudomonas (fluorescens), Lactobacillus, Streptomyces, Salmonella or Bacillus.

Typical mammalian host cell lines include, Hela, HEK293, H9, Per.C6 and Jurkat cells, mouse NIH3T3, NS0 and C127 cells, COS 1, COS 7 and CV1, quail QC1-3 cells, mouse L cells, mouse sarcoma cells, Bowes melanoma cells and Chinese hamster ovary (CHO) cells.

Also within the scope of the present invention are primary mammalian cells or cell lines. Primary cells are cells which are directly obtained from an organism. Suitable primary cells are, for example, embryonic fibroblasts (EF), primary hepatocytes, cardiomyocytes and neuronal cells as well as muscle stem cells (satellite cells), dermal and pulmonary fibroblasts, epithelial cells (nasal, tracheal, renal, placental, intestinal, bronchial epithelial cells), secretory cells (from salivary, sebaceous and sweat glands), endocrine cells (thyroid cells), adipose cells, smooth muscle cells, skeletal muscle cells, leucocytes such as peripheral blood mononuclear cell (PBMC), B-cells, T-cells, NK-cells, macrophages, neutrophils or dendritic cells and stable, immortalized cell lines derived thereof (for example hTERT or oncogene immortalized cells).

Appropriate culture media and conditions for the above described host cells are known in the art.

The host cells in accordance with this embodiment may, e.g., be employed to produce sufficient amounts of the fusion protein of the present invention suitable for detection.

The transfer of the nucleic acid molecule of the first aspect of the invention into the cell of the invention can be established, for example, by: i) CRISPR/Cas 9 mediated targeted genome integration after plasmid transfection or nucleofection, ii) retroviral gene transfer with stable random integration into the genome or iii) adenoviral or adeno-associated viral vectors that lead to transient gene expression without genomic integration.

In accordance with a preferred embodiment of the fourth aspect of the invention the cell is a lymphocyte, preferably a T-cell, more preferably a human T-cell.

A lymphocyte is a subtype of a white blood cell. Lymphocytes include natural killer cells (which function in cell-mediated, cytotoxic innate immunity), T-cells (for cell-mediated, cytotoxic adaptive immunity), and B-cells (for humoral, antibody-driven adaptive immunity).

The T-cells may be CD4 T-cells, CD8 T-cells or both and are preferably both. In initial T-cell therapies CD8 T-cells were primarily administered. These cells had optimal cytotoxicity but did not have sufficient replicative capacity after infusion, and with rare exceptions, the infused T-cell products had poor persistence in the patients (DeRenzo and Gottschalk, 2019, Shah and Fry, 2019, Labanieh et al., 2018). It is now widely accepted that mixtures of CD4 and CD8 T-cells are often preferred, likely because the CD4 T-cells provide growth factors and other signals to maintain function and survival of the infused CD8 T-cells.

In accordance with another preferred embodiment of the fourth aspect of the invention the cell further comprises a chimeric antigen receptor or a transgenic T-cell receptor.

T-cells are used for therapy in humans and in particular in anti-cancer treatments (Miliotou and Papadopoulou, 2018).

The currently most frequently used type of T-cell therapy is a Chimeric Antigen Receptor (CAR) T-cell therapy type of treatment in which a patient's T-cells (a type of immune system cell) are changed in the laboratory, so that they will attack cancer cells independent of the specificity of its T-cell receptor (DeRenzo and Gottschalk, 2019, Shah and Fry, 2019, Labanieh et al., 2018). In brief, T-cells are taken from a patient's blood. Then the gene for a special receptor that binds to a certain protein on the patient's cancer cells, often incorporating an scFv antibody fragment directed against a tumor antigen, is added in the laboratory. The special receptor is called a chimeric antigen receptor (CAR). Large numbers of the CAR T-cells are grown in the laboratory and given to the patient by infusion. CAR T-cell therapy is also called chimeric antigen receptor T-cell therapy in the art. Clinical trials have shown very promising results in end-stage patients with a full recovery of up to 92% in Acute Lymphocytic Leukemia (Miliotou and Papadopoulou, 2018). The transgenic T-cell receptor is generally comprised in a T-cell receptor modified T-cells.

The present invention relates in a fifth aspect to a kit comprising (i) the nucleic acid molecule, the vector, the fusion protein, and/or the cell of the above aspects of the invention, and (ii) an exogenous ligand.

The definitions and preferred embodiments of the above aspects of the invention apply mutatis mutandis to the fifth aspect of the invention.

In particular, also in connection with the kit, the exogenous ligand preferably comprises a radionuclide, wherein the radionuclide is preferably selected from C-11, F-18, Sc-44, Sc-47, Cu-64, Ga-68, Y-86, Y-90, Zr-89, Tc-99m, In-111, I-123, I-124, I-131, Tb-152 and Lu-177.

Yet further in accordance with a preferred embodiment the exogenous ligand is a small molecule, wherein the small molecule is preferably selected from (i) a chelator, preferably a metal chelator complex, more preferably [(R)-2-amino-3-(4-aminophenyl)propyl]-trans-(S,S)-cyclohexane-1,2-diaminepentaacetic acid (p-NH₂-Bn-CHX-A″-DTPA), (ii) an alkaloid, preferably colchicine or a demethylated colchicine, (iii) an iron-chelating siderophore, preferably petrobactin, enterobactin or bacillibactin, (iv) a plant steroid, preferably digoxigenin or digitoxigenin, (v) an organic dye, preferably fluorescein or aminofluorescein, or the exogenous ligand is a peptide, preferably a peptide with at least 2 and less than 10 amino acid residues, preferably VFFAED (SEQ ID NO: 29). Particularly preferred examples of small molecules are shown in FIG. 13A to E, wherein the small molecule as shown in FIG. 13C is most preferred.

The various components of the composition may be packaged as a kit with instructions for use. For instance, the components of the kit may be packed into one or more vials. The instructions for use preferably provide guidance for using the kit in accordance with the sixth aspect of the invention.

The present invention relates in a sixth aspect to the cell of the fifth aspect for use in the treatment of a disease by a cell-based therapy or a gene therapy, preferably a T-cell based therapy.

The definitions and preferred embodiments of the above aspects of the invention apply mutatis mutandis to the sixth aspect of the invention.

A cell-based therapy is any treatment of prevention of a disease comprising administering a cell of a patient. The cell-based therapy is preferably a T-cell based therapy as has been described herein above. For instance, in the case of CAR T-cell therapy a cell of the fifth aspect is used which expresses in addition the CAR. An example of a cell-based therapy is a therapy in which a viral vector is used in order to temporarily express a gene of interest in a cell type in order to treat the disease phenotype. This may be a growth factor, a shRNA to knock down a gene or a CRISPR/Cas 9 pair that changes the genome.

Gene therapy is designed to introduce genetic material into cells to compensate for an abnormal gene or to produce a beneficial protein. For instance, if a mutated gene causes a necessary protein to be faulty or missing, gene therapy may be able to introduce a normal copy of the gene to restore the function of the protein. In the case of a gene therapy the cell to be used in the therapy is therefore the cell of the fifth aspect, wherein the cell in addition expresses the genetic material to compensate for an abnormal gene or wherein the cell produces the beneficial protein.

The technical advantage of using a cell of the fifth aspect in the treatment of a disease by a cell-based therapy or a gene therapy, preferably a T-cell based therapy and, hence, a cell of the fifth aspect as the therapeutic cell is that upon the administration of an exogenous ligand as described herein above the location of the therapeutic cell in the patient or subject to be treated can be determined. This will be explained in more detail in connection with the following seventh aspect of the invention.

The present invention relates in a seventh aspect to an exogenous ligand for use in an in vivo method of diagnosing the efficacy of a cell-based therapy, preferably a T-cell based therapy in a subject, wherein the subject has been treated with the cell of the fifth aspect, and wherein the method preferably comprises positron emission tomography (PET) or single photon emission computed tomography (SPECT).

The definitions and preferred embodiments of the above aspects of the invention apply mutatis mutandis to the seventh aspect of the invention.

A positron emission tomography (PET) scan is inter alia used as a diagnostic imaging that can help to reveal the activity of therapy in certain tissues and organs. For this imaging, a PET scanner detects the presence of a radionuclide whose positron emission leads to a pair of perpendicularly photons with opposite directions, each with energy of 511 keV. Single-photon emission computed tomography (SPECT, or less commonly, SPET) is a nuclear medicine tomographic imaging technique using gamma rays. It is very similar to conventional nuclear medicine planar imaging using a gamma camera (that is, scintigraphy) but is able to provide true 3D information. Hence, PET and SPECT both require the administration or the delivery of a radioisotope (a radionuclide) into the patient, preferably through injection into the bloodstream. As described herein above, the exogenous ligand according to the invention may comprise such a radionuclide.

Hence, also in accordance with the seventh aspect the exogenous ligand preferably comprises a radionuclide, wherein the radionuclide is preferably selected from C-11, F-18, Sc-44, Sc-47, Cu-64, G-68, Y-86, Y-90, Zr-89, Tc-99m, In-111, I-123, I-124, I-131, Tb-152 and Lu-177.

Further in accordance with a preferred embodiment the exogenous ligand is a small molecule, wherein the small molecule is preferably selected from (i) a chelator, preferably a metal chelator complex, more preferably [(R)-2-amino-3-(4-aminophenyl)propyl]-trans-(S,S)-cyclohexane-1,2-diaminepentaacetic acid (p-NH₂-Bn-CHX-A″-DTPA), (ii) an alkaloid, preferably colchicine, (iii) an iron-chelating siderophore, preferably petrobactin, enterobactin or bacillibactin, (iv) a plant steroid, preferably digoxigenin or digitoxigenin, (v) an organic dye, preferably fluorescein or aminofluorescein, or the exogenous ligand is a peptide, preferably a peptide with at least 2 and less than 10 amino acid residues, preferably VFFAED (SEQ ID NO: 29). Particularly preferred examples of small molecules are shown in FIG. 13A to E, wherein the small molecule as shown in FIG. 13C is most preferred.

Unless otherwise defined, all technical and scientific terms used herein have the same meaning as commonly understood by one of ordinary skill in the art to which this invention belongs. In case of conflict, the patent specification, including definitions, will prevail.

All amino acid sequences provided herein are presented starting with the most N-terminal residue and ending with the most C-terminal residue (N to C), as customarily done in the art, and the one-letter or three-letter code abbreviations are used to identify amino acids throughout the present invention correspond to those commonly used for amino acids.

Regarding the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims.

The figures show:

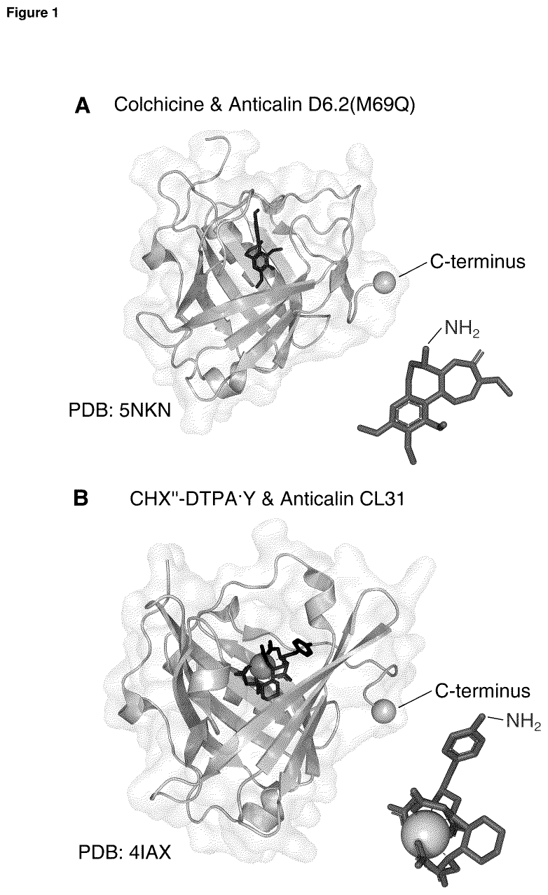

FIG. 1: Crystal structure of two Anticalin⋅ligand pairs for use in the invention. (A) Crystal structure of the Anticalin D6.2 (M69Q) in complex with colchicine (PDB ID: 5NKN). (B) Crystal structure of the Anticalin CL31 in complex with Tris-CHX-A″-DTPA⋅⁸⁹Y (PDB ID: 4IAX). In both protein structures the C-terminus of the lipocalin-based binding protein, which is fused with the V5-tag and/or the transmembrane domain, is highlighted. Furthermore, the unique primary amino-group within the ligand moiety, which can be conjugated to a linker and a functional moiety, is highlighted.

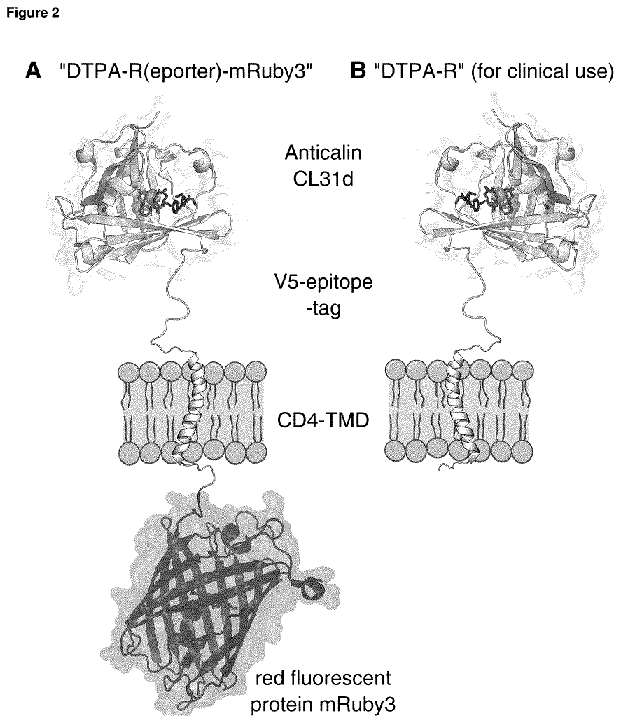

FIG. 2: Schematic representation of the membrane-associated PET-reporter proteins. (A) The reporter protein intended for preclinical development comprising an Anticalin featuring a binding activity for DTPA, a V5-tag, a CD4 transmembrane domain and a cytoplasmic mRuby3 fluorescent protein. (B) Schematic representation of a similar reporter protein without a fluorescent protein (intended for clinical use).

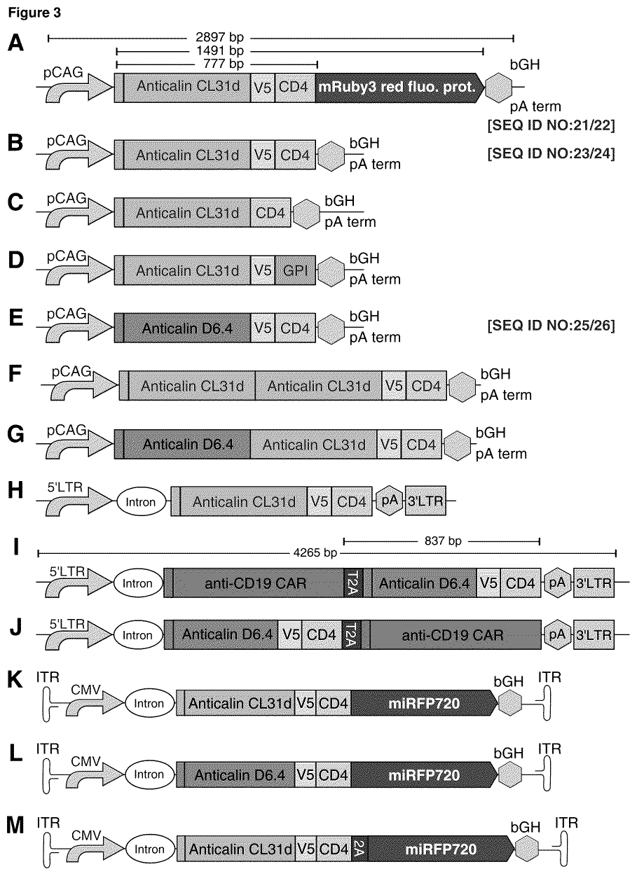

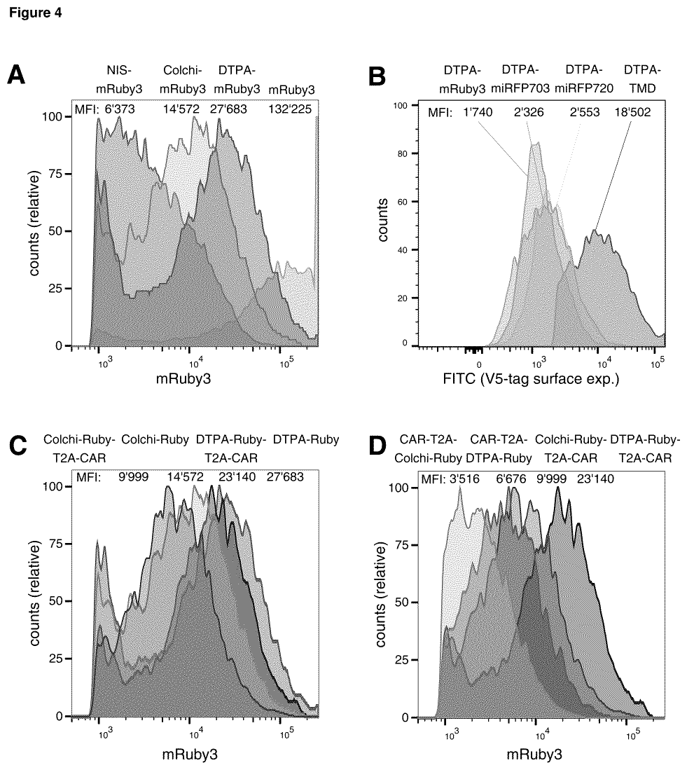

FIG. 3: Modular DNA- and Protein-design of the reporter protein. The various expression cassettes are composed of regulatory and coding regions which are both assembled from different elements. Upstream regulatory elements are promoters that initiate transcription of the open reading frame (ORF), such as the Chicken Actin Promoter (CAG), the 5′ Long Terminal Repeats (5′LTR) or the Cytomegalovirus promoter (CMV) together with introns for increased expression strength. Downstream of the ORF, polyadenylation signals are incorporated for optimal protein expression. Furthermore, with viral vector systems genetic elements for amplification and packaging are incorporated such as the 5′LTR/3′LTR of Lentivirus or the Inverted Terminal Repeats (ITRs) of the Adeno-associated Virus (AAV). The reporter protein is composed of a secretory signal sequence/signal peptide for the incorporation into the membrane, one or more lipocalin-derived binding proteins that serve as binding domain for the radioligand, an optional V5-epitope tag and a transmembrane domain derived from human CD4 followed by an optional fluorescent protein. Furthermore, some expression constructs featured two different membrane proteins that are both expressed from a single mRNA, each having a secretory signal peptide for insertion into the membrane, just separated by a 2A self-cleaving peptide. A set of exemplary expression constructs that were constructed: (A) Full reporter gene with red fluorescent protein (“preclinical DTPA-R”), (B) without fluorescent protein (“clinical DTPA-R”), (C) without V5-tag, (D) with sequence for the GPI-anchor amidation of e.g. a serine residue instead of a transmembrane domain, (E) with a colchicine binding Anticalin (called “Colchi-R”), (F) a homo-bivalent reporter gene, (G) a hetero-duovalent reporter gene, (H) a reporter gene expression cassette for the use of lentiviral transduction technique, (I) a T2A-separated expression cassette for a Chimeric Antigen Receptor (CAR) and a reporter gene, (J) the same construct as before with inverted orientation, (K-L) a reporter protein with miRFP720 as fluorescent protein that features ITRs for gene transfer of the reporter gene using recombinant AAV viral vectors. (M) Example of a reporter protein that features an intracellular fluorescent protein which is not connected to the cell membrane by a polypeptide chain but separated by a self-cleaving 2A sequence. Intracellular self-cleavage and separation of the fluorescent protein leads to much higher cell surface expression of the membrane-anchored reporter protein compared to the situation where the fluorescent protein is permanently fused to the reporter protein (see, e.g., FIG. 4B).

FIG. 4: Determination of expression levels obtained for different reporter proteins and reporter gene expression cassettes by flow cytometry. Lentiviral vectors were produced and used for the infection of the human T-cell line Jurkat. Cells were analyzed after infection using flow cytometry on a LSRFortessa (BD Biosciences) for intrinsic mRuby3 fluorescence or the V5-tag presented on the cell surface. (A) Expression levels of different reporter genes. Sodium/iodide symporter fused to mRuby3 (NIS-mRuby3) and a soluble mRuby3 protein were compared to the reporter proteins of the invention, that is DTPA-R and Colchi-R, both fused to mRuby3. (B) Comparison of different fluorescent proteins fused to the C-terminus of the reporter protein DTPA-R and located in the cytoplasm. Cells were stained with an Alexa Fluor 488-conjugated antibody (SV5-PK1) directed against the extracellular V5-tag. (C) Comparison of reporter proteins without or together with a chimeric antigen receptor (CAR) fused via a C-terminal 2A-sequence on a single mRNA. (D) Comparison of inverted order of reporter protein and CAR for DTPA-R and Colchi-R.

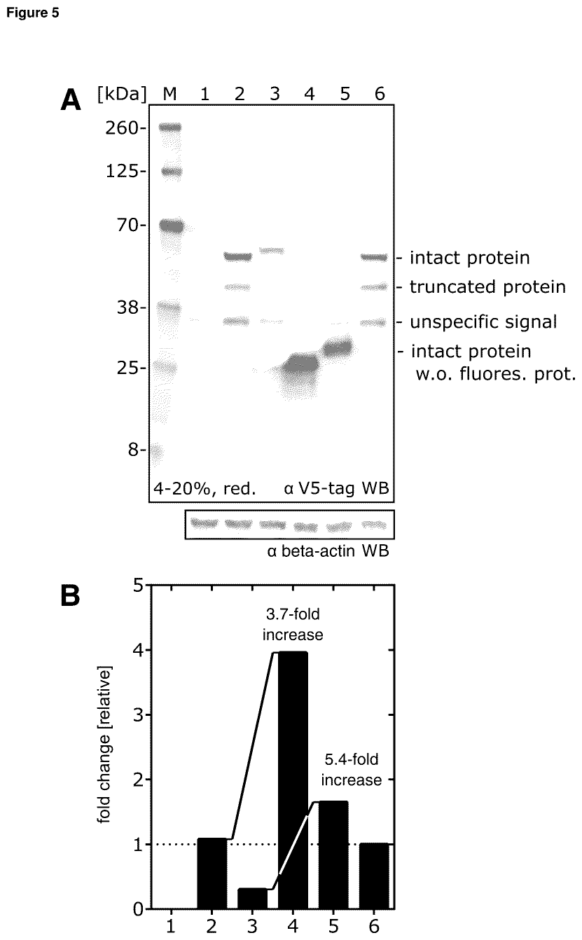

FIG. 5: Western blot analysis of cells expressing the reporter proteins of the invention. The human T-cell line Jurkat was modified for expression of the reporter proteins of the invention using lentiviral transduction or CRISPR/Cas9 mediated gene transfer. Corresponding cells were cultivated, harvested, and proteins were extracted using RIPA-buffer (Thermo Scientific). Samples were adjusted for the same total protein amount and separated using SDS-PAGE (4-20% under reducing conditions), followed by blotting onto a PVDF membrane. The membrane was incubated with the monoclonal anti-V5-antibody SV5-PK1 and appropriate fluorescent secondary reagents (800 nm channel), together with a control antibody recognizing beta-actin (DyLight680-AbD12141, Bio-Rad). Signals were detected using an Odyssey scanner (LI-COR) (A) and signals were analyzed using LI-COR software, which allowed quantitative comparison of the individual reporter proteins (B). The following samples were analyzed: 1) Jurkat cell line without transgene, 2) Jurkat DTPA-V5-TMD-mRuby3, 3) Jurkat Colchi-V5-TMD-mRuby3, 4) Jurkat DTPA-V5-TMD, 5) Jurkat Colchi-V5-TMD, 6) Jurkat DTPA-V5-TMD-mRuby3 “clone Jurkat 7-14”. While sample 1) was a non-transgenic mock sample/control, 2-5) were lentivirally transduced cell lines and 6) was stably modified using CRISPR/Cas 9 plasmid transfection and subsequent selection using appropriate antibiotics. Besides the intact protein corresponding to the complete reporter protein with processed signal peptide, a truncated protein species is present, in which the reporter protein has been proteolytically cleaved, most probably within the intracellularly located fluorescent protein domain. The detection of cell lysates using the B1 antibody also results in an unspecific band produced by a cross-reactivity of the antibody with an unknown host protein, which is also present in the HEK cells that were not genetically modified.

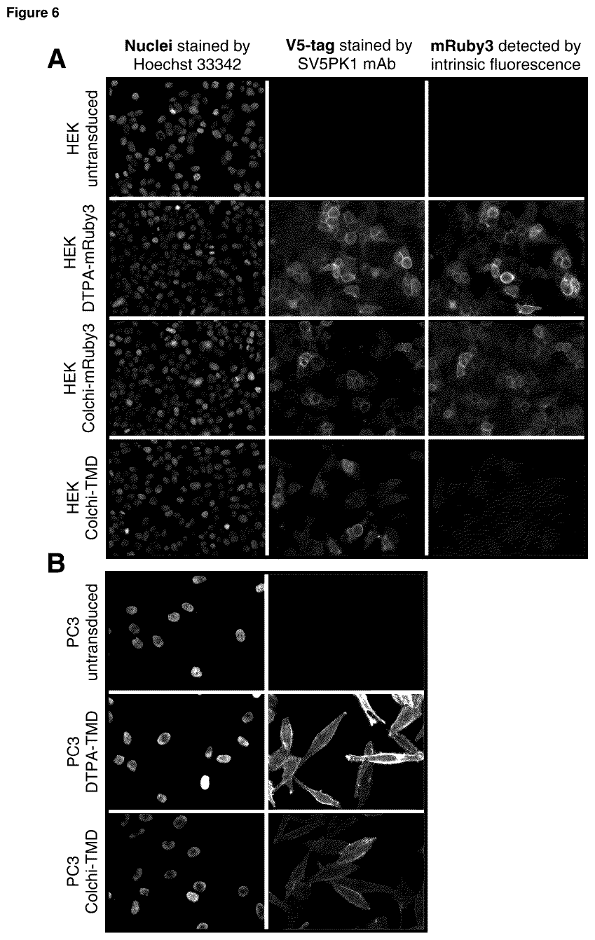

FIG. 6: Fluorescence microscopy of cells expressing the reporter proteins of the invention. (A) HEK (humand embryonic kidney) or (B) PC3 (human prostate carcinoma) cells were transduced with lentiviral vectors to express different versions of the DTPA-R or the Colchi-R, subjected to FACS cell sorting and subsequently grown in 96-well plates, then stained with Hoechst 33342 and the anti-V5-tag antibody SV5-PK1 as well as the appropriate secondary reagent, and finally imaged using an Evos M7000 fluorescence microscope (Thermo Scientific) with appropriate filters at 40-fold magnification.

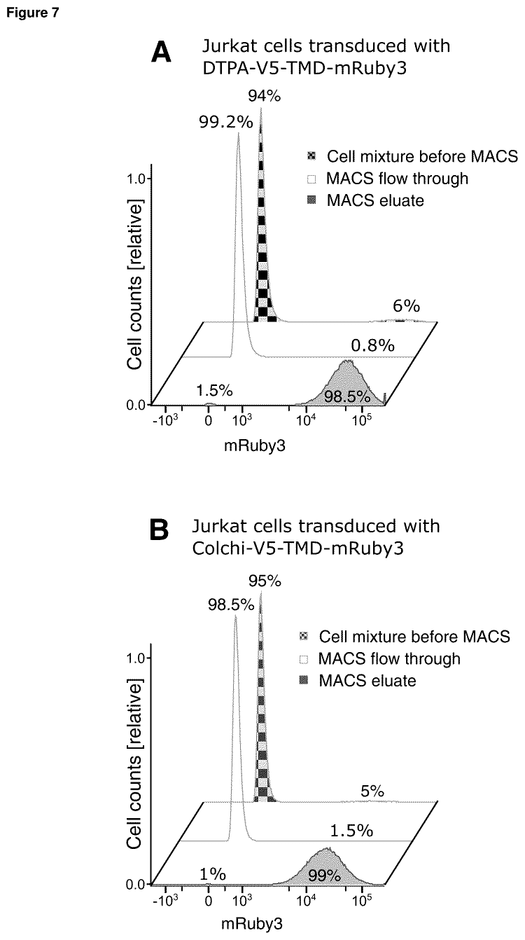

FIG. 7: Magnetic-activated Cell Separation (MACS) using the reporter protein of the invention. Jurkat cells expressing the DTPA-R (A) or the Colchi-R (B) were mixed with un-transduced Jurkat cells at a 5:95 ratio. A total of 10×10⁶ cells were incubated for 30 min on ice with 2 μg anti-V5-tag antibody SV5-PK1 (Bio-Rad Laboratories). Subsequently, 20 μl anti-mouse IgG (H+L)-microbeads were added and binding was allowed for 15 min at 4° C. Then, the cell suspension was loaded onto a MiniMACS separation column (type MS) which was magnetized using a MiniMACS separator magnet (all from Miltenyi Biotec). After washing with MACS buffer, the column was removed from the magnetic field and the cells of interest were eluted together with the MACS buffer. Finally, a sample of the initial cell suspension the flow through and the eluted cell fraction were analyzed using flow cytometry.

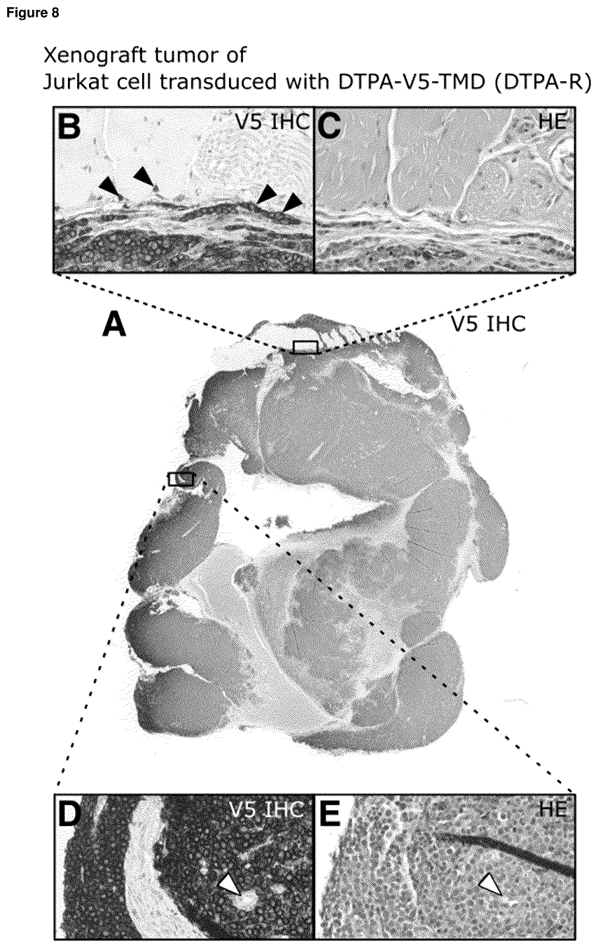

FIG. 8: Detection of cells tagged with the invention on a cellular level using immunohistochemistry.

Jurkat cells, either transfected with DTPA-R or Colchi-R were injected s.c. into CD1 nude mice (Charles River Laboratories) and explanted after mice had been sacrificed. Tumor tissue was fixed in 10% (w/v) neutral-buffered formalin solution (Otto Fischar, Saarbrücken, Germany) for 48 h and stored in PBS at 4° C. Tissue samples were dehydrated using an automated system (ASP300S; Leica Biosystems) and subsequently embedded in paraffin. Serial 2 μm sections were prepared with a rotary microtome (HM355S; ThermoFisher Scientific) and subjected to histological and immunohistochemical analysis. Hematoxylin and eosin (H&E) staining was performed on deparaffinized sections with Eosin and Mayer's Haemalaun (Morphisto, Frankfurt am Main, Germany). Immunohistochemistry was performed using a Bond RXm system (Leica Biosystems) with primary antibodies against V5-tag (SV5-Pk1; Biorad) using 1:250 dilutions. Briefly, slides were deparaffinized using deparaffinization solution (Leica Biosystems), pretreated with Epitope retrieval solution 1 (corresponding to citrate buffer, pH 6) for 30 min. Bound antibody was detected with a polymer refine detection kit without post primary reagent and with an intermediate Rabbit anti-mouse IgG secondary antibody (diluted 1:400; Leica Biosystems) and signals were developed with 3,3′-diaminobenzidine (DAB). Representative images were collected on an Aperio AT2 digital pathology slide scanner using ImageScope (ver.12.3) software (both from Leica Biosystems).

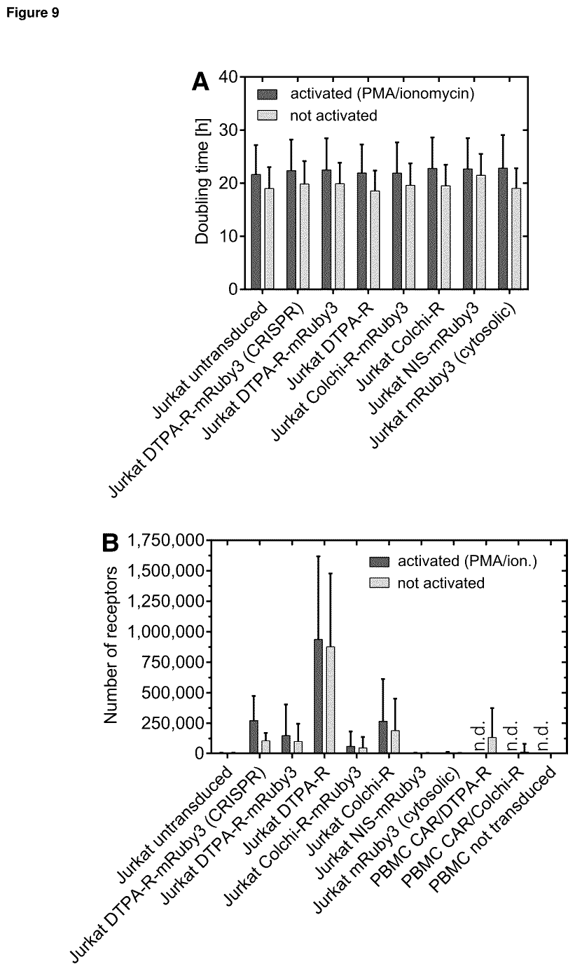

FIG. 9: Determination of expression levels of the reporter proteins in T-cells and the influence on the cell division rate.

The human T-cell line Jurkat was lentivirally transduced with expression cassettes for different reporter proteins and after FACS sorting of the 10% highest expressing clones, individual stable sub-cell lines were created. For these cell lines the doubling time was assessed using the CFSE assay (A) and the number of receptors on the cell surface was quantified using flow cytometry measurements (B).

To this end, cells were cultivated in RPMI medium with 10% FCS and penicillin and streptomycin. In order to further assess the influence of the T-cell activation on cell division rate and reporter protein expression, the cells were unspecifically activated with Phorbol myristate acetate (PMA) and lonomycin (both from InvivoGen, Toulouse, France). Cells were seeded at a density of 0.5×10\hat{ }6 cells/ml in medium with or without 1 μg/ml PMA and 10 μg/ml lonomycin for 24 h and were then transferred into medium without chemical activators and measurements were performed after 2 days. (A) Exponentially dividing Jurkat cells were counted and a number of 15×10\hat{ }6 cells was dissolved in 500 μl PBS and labeled with the CFSE Cell Division Tracker Kit (BioLegend, CA, USA) for 20 min at 37° C. The fluorescent probe 5-(and 6)-Carboxyfluorescein diacetate succinimidyl ester (CFSE) conjugates to primary amine groups on the cells which subsequently can be quantified using flow cytometry. As the CFSE-signal of cells is reduced by 50% by every cell division, the quantification of the signal after some days allows calculation of the number of cell division that had occurred. In order to quantify the loss of signal, in addition to the stained cells also beads with a known concentration of the same fluorescent dye (Quantum FITC-5 MESF, Bangs Laboratories) were used for flow cytometry on a LSRFortessa (BD Biosciences) instrument. The reduction of the fluorescence signal was analyzed following 4 days of cell growth and the MESF kit was then used to calculate the number of corresponding fluorescein dyes that was then used to calculate the doubling time. (B) The same cells, with and without chemical activation using PMA/lonomycin were counted and a number of 0.5*10\hat{ }6 cells was stained with 2 μl of a 147 μg/ml SV5-PK1 antibody solution (Bio-Rad Laboratories, Hercules, CA) that had been conjugated with Alexa Fluor488-NHS (Lumiprobe, Hannover, Germany) at a degree of labeling of 3.85 dyes per antibody. Here the Quantum Alexa Fluor 488 MESF kit (Bangs Laboratories, Fishers, In) was used to calculate the number of Molecules of Soluble Fluorochrome (MESF) based on mean fluorescence intensities and it was assumed that one antibody binds with its two paratopes (Fab arms) to two reporter proteins. Error bars indicate standard deviation and represent the variance of the cell populations recorded in flow cytometry.

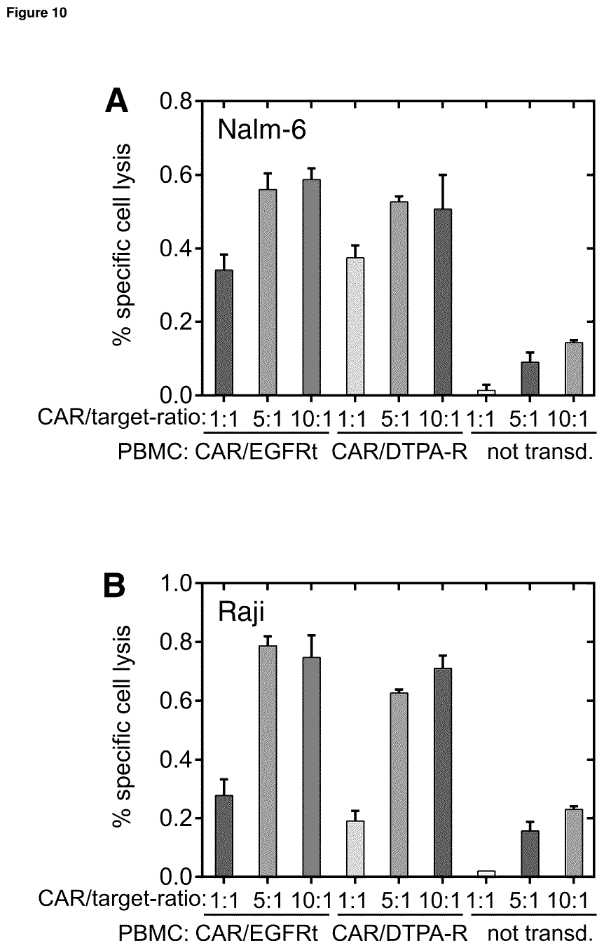

FIG. 10: Absence of negative influence of the reporter protein on the cellular function of a CAR T-cell

In order to confirm the absence of negative impact of the reporter protein of the invention on the cellular function of a CAR T-cell, two different expression constructs were compared, both featuring an anti-CD19 chimeric antigen receptor, a self-cleaving 2A peptide followed by either a truncated version of the epidermal growth factor receptor (EGFRt) or the reporter protein DTPA-R. In short, peripheral blood mononuclear cell (PBMC) were isolated from a healthy donor and subsequently transduced with lentiviral vectors encoding the described expression cassettes (CAR/EGFRt or CAR/DTPA-R). After isolation of the cell population expressing the transgene, cells were expanded in RPMI medium with 200 U/mI IL-2, 12.5 ng/ml IL-7 and IL-15, each (all from PeproTech, Rocky Hill, NJ, USA) and maintained at a cell density of 0.25×10⁶ cells/ml. The ability of the CAR T-cells to kill CD19 expressing target cells was assessed by Chrome-51 (⁵¹Cr) release assay that works by loading the target cells with the radioactive isotope and the subsequent quantification of the Cr-51 that has been released into the supernatant of the culture. For this assay, the CD19-positive target cell lines Nalm-6 (A) or Raji (B) were incubated together with the respective CD19-CAR T-cells at different ratios of T-cells over target cells. A number of 10 000 target cells was incubated with the respective number of PBMCs in a V-bottom 96-well plate in 150 μl culture medium. The plate was incubated for 4 h with occasional agitation at 37° C. Afterwards, the radioactivity in the supernatant was quantified using a Wizard2 gamma counter (PerkinElmer) and the Percent Specific Lysis was calculated using the following formula: [(experimental release−spontaneous release)/(maximum release−spontaneous release)]*100. In addition, the ability of the CAR T-cells to kill CD19 expressing target cells was assessed by a FACS killing assay (C-F). To this end, GFP expressing NALM-6 target cells were incubated with 1:1 or 5:1 excess of CAR/DTPA-R or CAR/EGFRt CAR T-cells. After 48 h a comparable fraction of the cultured cells (as measured by FACS quantification beads) was analyzed and the number of GFP-positive target cells that remained after the killing assay was quantified.

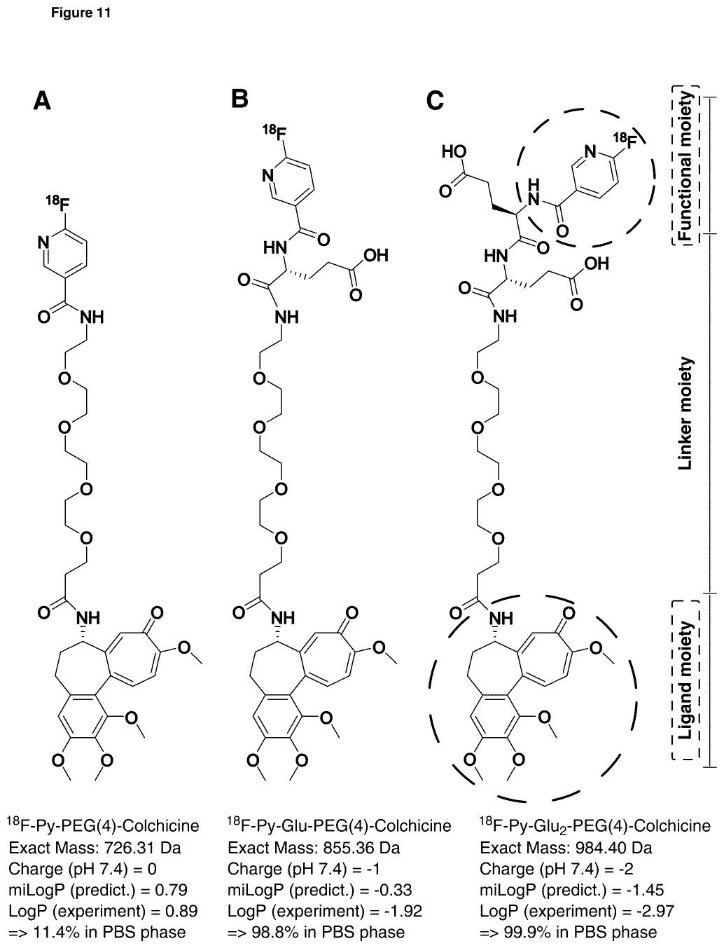

FIG. 11: Composition of an exemplary ¹⁸F-labeled radioligand composed of a ligand moiety (here colchicine, alternatively Bn-CHX-A″-DTPA), a PEG linker and a radiolabel moiety (e.g. radio-fluorinated pyridine for PET-detection). Hydrophilicity optimization of a fluorine-18 labeled radioligand used in conjunction with the Colchi-R. In this example, the radioligand composed of the colchicine ligand moiety, a PEG-linker and a fluorinated pyridine was analyzed (A), resulting in an unfavorable LogP value that indicated low hydrophilicity. Of note, hydrophilicity is crucial to prevent unspecific accumulation in cells or cell membranes and to ensure favorable distribution in the body as well as subsequent elimination by the renal route. Thus, two derivatives with increased hydrophilicity were designed featuring one (B) or two (C) interspersed D-Glu residues carrying negatively charged side chains. The incorporation of one D-Glu residue shifted the LogP value to −1.92 (meaning 98.8% in the PBS phase) and the additional incorporation of a second D-Glu residue further increased the LogP value to −2.97 (which means 99.9% of the radioligand is distributed in the aqueous PBS phase) rendering it a promising radioligand candidate.

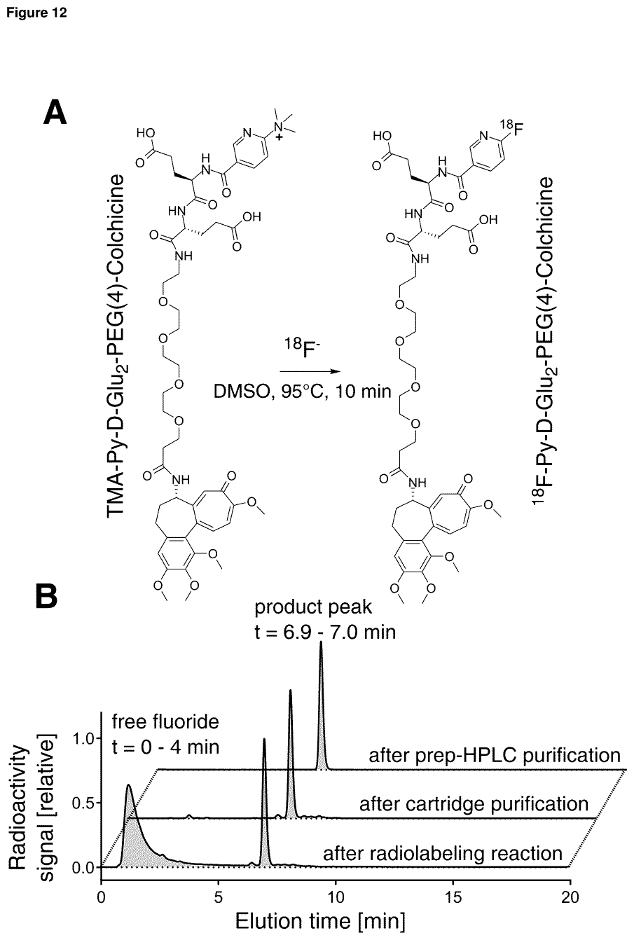

FIG. 12: Labeling reaction, quality control and in vivo biodistribution for ¹⁸F⁻ for ¹⁸F-Py-Glu₂-PEG(4)-Colchicine.

The precursor ⁺TMA-Py-D-Glu-D-Glu-PEG(4)-Colchicine was used for synthesis of ¹⁸F-Py-D-Glu-D-Glu-PEG(4)-Colchicine (called ¹⁸F-Colchicine). (A) In short, the radioactive ¹⁸F⁻ was produced by a cyclotron in house. Fluorine was eluted from a Sep-Pak Accell Plus QMA Carbonate (Waters) cartridge using 75 mM Tetrabutylammonium (TBA HCO₃) solution and was subsequently dried in a Wheaton V-Vial under a continuous flow of dry argon gas at 95° C. by azeotropic distillation. An amount of 0.5 to 2 mg of the precursor was dissolved in DMSO and added into the V-Viral. The exchange reaction of ¹⁸F for the Trimethyl ammonium (TMA) leaving group also occurred at 95° C. for 10 minutes. Subsequently, the reaction mixture was purified by a Chromafix PS-H⁺ (Macherey-Nagel) and a Sep-Pak C18 Classic (Waters) cartridge, which allows the quantitative removal of the unlabeled precursor by its positive charge. Subsequently, the activity was eluted from the C18 cartridge by EtOH and in some cases the radioligand was further purified by HPLC with a Multospher 100 RP 18−5μ 5μ 250×10 mm column with an isocratic elution at 25% Acetonitrile with 0.1% TFA.

(B) Quality control by HPLC of the radiosynthesis of ¹⁸F-Py-D-Glu-D-Glu-PEG(4)-Colchicine: i) after radiolabeling reaction, ii) after cartridge purification and iii) after preparative HPLC purification, which were performed on a 100×4.6 mm Chromolith performance RP-C18 column (Merck Millipore) with a gradient of 5 to 55% acetonitrile with 0.1% TFA at a flow rate of 3 ml/min. A single peak was obtained as for both other precursors shown in FIG. 9 indicating a pure radioligand.

FIG. 13: Modular chemical design of radioligands for the DTPA-R

Based on the required ligand moiety for the DTPA-R (here NH₂-Bn-CHX-A″-DTPA) there are different variants of radioligands that can be synthesized and compared in order to find the lead candidate which is expected to exhibit high affinity for the DTPA-R, in vivo stability and the absence of quantitative structural modifications in the patient and a favorable excretion pattern (ideally only via the renal route).

(A) The ligand moiety alone can serve as the radioligand when used together with an appropriate radiometal. (B) A variant where the amino-group within the NH₂-Bn-CHX-A″-DTPA was replaced by the leaving group for radiofluorination (trimethylamine leaving group according to the “minimalist approach for radiofluorination” (Richarz et al., 2014)). (C) A design with a radiofluorination moiety, two D-Glu residues and a PEG(4) linker (called ¹⁸F-DTPA) as in FIG. 11-12 for ¹⁸F-Colchicine. (D-E) A bivalent radioligand with two ligand moieties which allows the loading with radioactive and non-radioactive metals. By the bivalent binding of the radioligand to the cell of interest via two different Anticalins, the overall affinity of the radioligand is drastically increased compared to a monovalent radioligand (comparable to the bivalent binding of full-length antibodies to cells). Dimerization was accomplished by a homo-bifunctional PEG(13)-NHS₂ (D) or a aliphatic linker with interspaced peptide bonds (E).

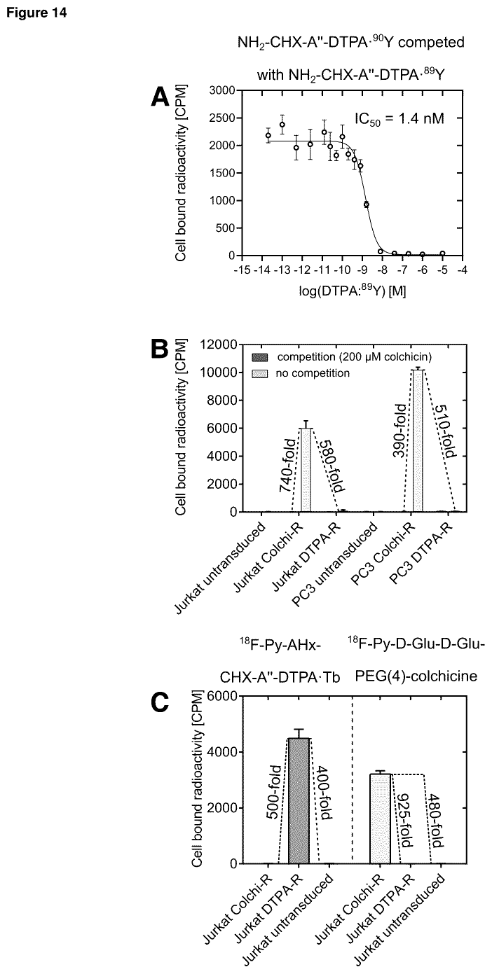

FIG. 14: Confirmation of radioligand binding to cells tagged with the invention using radioligand binding assays.

The binding of different radioligands to cells that express the DTPA-R or Colchi-R was shown in binding assays. To this end, Jurkat or PC3 cells that stably express the respective reporter protein of the invention were cultivated, counted and a defined concentration was incubated together with the radioligand in PBS with 2% BSA for 1 h at 37° C. Subsequently, the cells were washed twice with PBS with 2% BSA and finally in the cell-bound fraction and the supernatant fraction the respective radioactivity was detected. (A) NH₂-Bn-CHX-A″-DTPA⋅Y-90 binding was competed with a non-radioactive competitor NH₂-Bn-CHX-A″-DTPA⋅Y-89. (B) Specific binding of ¹⁸F-Py-D-Glu-D-Glu-PEG(4)-Colchicine to Colchi-R Jurkat and Colchi-R PC3 cells was quantified and compared to the reporter gene with another specificity (DTPA-R). Furthermore, the specific binding was blocked by 200 μM Colchicine solution to prove the specificity of the binding. (C) A comparative binding assay with ¹⁸F-Py-Ahx-DTPA and ¹⁸F-Py-Glu-Glu-PEG(4)-Colchicine to Jurkat cells expressing either DTPA-R or Colchi-R proving the orthogonal functionality of the two reporter protein & reporter probe pairs.

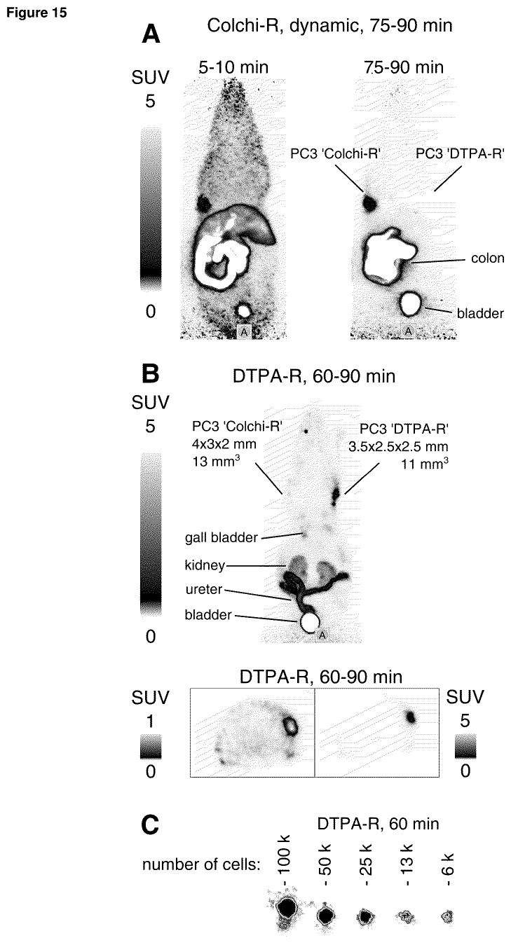

FIG. 15: In vivo PET-imaging of subcutaneous PC3 xenograft tumors tagged with the invention.

CD1 nude mice were subcutaneously injected with a Colchi-R PC3 xenograft in the right flank and a DTPA-R PC3 xenograft in the left flank. After xenograft tumors had developed the fluorine labeled radioligand was injected i.v. and a dynamic PET/MR was recorded for 90 minutes using a Mediso nanoScan PET/MR scanner. (A) Maximal intensity projections after ¹⁸F-Py-D-Glu-D-Glu-PEG(4)-Colchicine i.v. injections are shown for the 5-10 min and 75-90 min time frame and exhibit a hepato-biliary excretion pattern. (B) In contrast, the i.v. injection of ¹⁸F-Py-D-Glu-D-Glu-PEG(4)-DTPA⋅Tb (¹⁸F-DTPA) resulted in the specific accumulation of the radioligand in the tumor at the opposite shoulder (PC3 DTPA-R) together with a favorable, exclusively renal excretion pattern. Here, the mouse was awake for 60 min ahead of the PET-scan that was recorded von 60-90 min post injection. For the successful detection of this small xenograft tumor, also axial image sections are depicted. (C) Furthermore, Jurkat cells expressing DTPA-R were incubated in vitro with an excess of ¹⁸F-DTPA and washed twice. Subsequently, a dilution series was prepared and the cells were scanned within PCR tubes for 60 min to determine the detection limit of the reporter gene system, which was found to be around 5.000 cells.

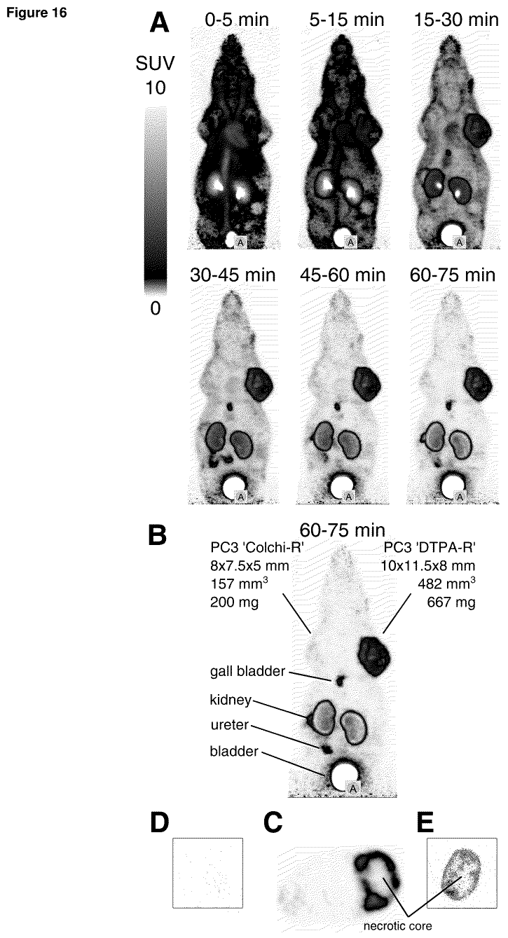

FIG. 16: Dynamic PET-imaging of DTPA-R xenograft tumor with ¹⁸F-DTPA⋅Tb

(A) Comparable to the PET-imaging study in FIG. 15 a mouse with larger PC3 DTPA-R and PC3 Colchi-R xenograft tumors on the right and left shoulder were dynamically scanned for 90 min after injection of ¹⁸F-DTPA. (B) at the time frame 75-90 min p.i. a remarkably low background signal and a pronounced tumor signal are visible. Additionally, there are elimination related signal in gall bladder, kidneys, ureter and the urine bladder. (C) an axial section through the tumor reveals a signal for the DTPA-R tumor and no signal above the background for the Colchi-R tumor. Furthermore, a necrotic core was visible in the sectional view of the PET which is characteristic for PC3 xenografts. (DE) The distribution of the radioligand at the border of the tumor and the absence of radioligand within the necrotic core was confirmed by autoradiography experiments.

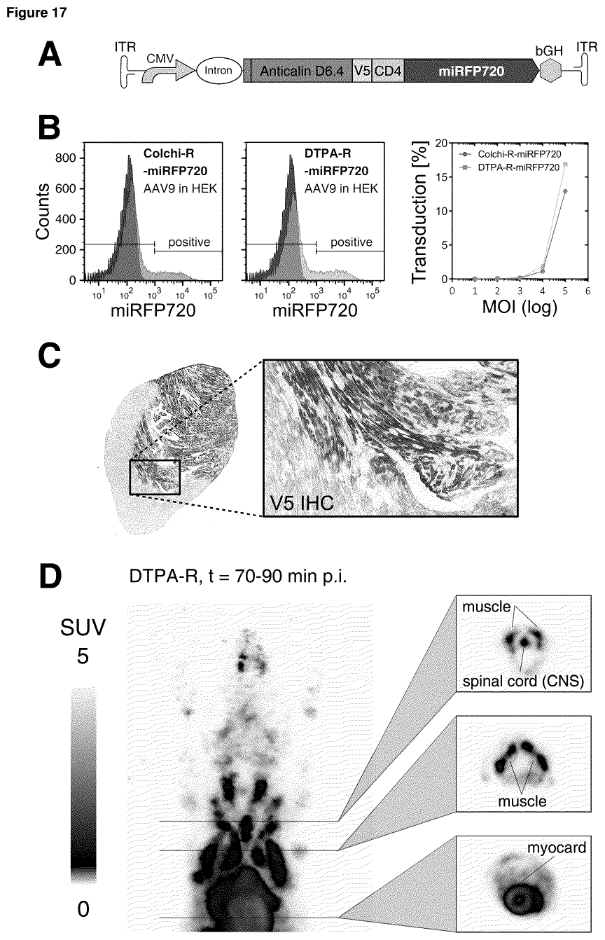

FIG. 17: Use of DTPA-R for the detection and quantification of AAV9 transduction events in vivo

(A) For the detection of viral transduction events based on the adeno-associated virus, viral vectors were constructed by flanking an expression cassette for the DTPA-R or the Colchi-R with AAV2 inverted terminal repeats (ITRs). This genetic construct was used to produce and purify AAV9 viral vectors. (B) A dose dependent transduction was confirmed for DTPA-R and Colchi-R reporter genes after transduction of HEK cells with respective AAV viral vectors. (C) Anti-V5 immunohistochemistry staining of an axial cryosection of a mouse heart that had been i.v. injected with AAV before. (D) Maximal intensity projection of a PET-scan of a mouse that had been injected 7 days before with AAV9 viral vectors encoding DTPA. Data was recorded 70-90 min post injection of the ¹⁸F-DTPA⋅Tb radioligand. Axial sections are depicted showing specific signal in shoulder and dorsal muscle tissue as well as the myocardium.

FIG. 18: Direct comparison of expression levels of an Anticalin in comparison with other binding proteins on the cell membrane

(A-C) Design of expression constructs for the comparison of Anticalin-based reporter proteins according to this invention versus scFv-based reporter proteins from the state of the art. (A) Construct for CL31d-V5-CD4TMD, (B) construct for scFv(muC825)-V5-CD4TMD and (C) construct for scFv(huC825)-V5-CD4TMD. Jurkat T-cells after stable retroviral transduction with one of the constructs were stained with an anti-VS-tag antibody conjugated with AlexaFluor488, which was detected using flow cytometry. (D) Histogram of the transduced cells analyzed by flow cytometry along with (E) the conversion to absolute numbers of receptors per cells (as explained for FIG. 9 herein above). From these stably transduced cell lines, the 10% highest expressing cells were isolated using Fluorescence Activated Cell Sorting (FACS). (F) Histogram of the FACS-sorted cells again analyzed by flow cytometry along with (G) the conversion into absolute receptor densities.

The following Examples illustrate the invention.

### EXAMPLE 1

### Development of a Novel Reporter Gene/Protein System

A reporter system was developed comprising a genetically encoded, membrane anchored reporter protein and a cognate small-molecule radioactive probe (radioligand) which can be injected intravenously. The radioligand is distributed in the blood compartment and selectively bound by the reporter protein, which is expressed on the genetically modified cell type. After rapid excretion of excess unbound radioligand via the kidneys or the hepato-biliary system, its radioactive decay in the body can be registered and by this way the distribution and location of the reporter gene-tagged cell type can be analyzed. This concept resembles the use of radiopharmaceuticals in nuclear medicine to detect various tumor targets (such as somatostatin, PSMA or integrins). The difference here is that the molecular target in the patient is a synthetic membrane-associated protein, whose coding gene has been incorporated specifically into the tagged cell type, which may serve for cell or gene therapy.

For an ideal reporter gene system, its components need to be bioorthogonal: neither the reporter (membrane) protein nor the radioligand should interfere with the healthy organs or tissues. This allows the monitoring of biological processes with minimal functional impairment and, on the other hand, with a minimal background signal. Furthermore, also the radioligand should be inert and not be modified or cleaved within the organism. A further requirement for clinical application is the lack of immunogenicity. For signal normalization, or for the simultaneous analysis of different biological processes, it is also desirable to have several independent reporter systems at hand that can be used in parallel without mutual interference (called multiplexing), similar to the well-established dual-luciferase systems or fluorescent proteins with distinct spectral properties used in biomedical research.

Anticalins are a class of engineered binding proteins based on the natural lipocalins. Most often, the human lipocalin 2 (Lcn2), also known as neutrophil gelatinase-associated lipocalin, NGAL, is used as scaffold for the selection of specific binding proteins (Richter et al., 2014, Schiefner and Skerra, 2015). The natural ligand of Lcn2 is the iron-chelating siderophore enterobactin that is employed by bacteria to sequester iron ions which are essential for their growth. The plasma protein Lcn2 binds the iron-siderophore-complex (Fe⋅enterobactin) before it can be taken up by the bacterium and, thus, restricts the growth of bacteria in the human body by deprivation of this essential metal. Lcn2 is part of the innate immune system and, in an engineered form, ideally suited for biomedical applications. Furthermore, the calyx-shaped ligand pocket of the lipocalins favors the tight binding of small-molecule ligands in correspondingly engineered Anticalins (FIG. 1).

The human origin of many Anticalins lowers immunogenicity after expression on a given cell of interest, e.g. an immune cell. As in current clinical investigations the immunogenicity of CAR receptors limits the persistence of some CAR T-cells in patients and hampers a positive therapy outcome, this is one of the most critical factors. Apart from protein or peptide targets, Anticalins have been selected to recognize several small molecules with high affinity (Table 1), for example petrobactin, a specific siderophore of certain Bacillus species (Dauner et al., 2018), colchicine (Barkovskiy et al., 2018) (FIG. 1A) and Bn-CHX-A″-DTPA (Kim et al., 2009) (Eggenstein et al., 2013) (FIG. 1B). These artificial ligands represent chemical structures foreign to human physiology (in contrast to the natural metabilites biotin, thymidine and analogues or iodine, which often have been used for cell tagging and, thus, may serve (after labelling) as bioorthogonal radioligands in the human body that are specifically recognized by the membrane-anchored Anticalin. The extraordinary affinity in the picomolar range of these Anticalins allows the imaging of the reporter construct and/or the transformed cell even at later time points after injection with a low background signal (due to the elimination of the free ligand). The high affinity of the Anticalin expressed on the cell surface can even be increased when a radioligand is used comprising two different ligand moieties, conjugated by an appropriate linker, that can be bound by two individual membrane-anchored Anticalins and, thus, result in an increased overall affinity (avidity effect, see FIG. 13 D-E for exemplary radioligands).

Generally, the reporter gene system of the invention features a modular design in a way that (i) different radionuclides can be incorporated into the radioligand via suitable chelator groups or selective conjugation chemistry to allow PET and/or SPECT imaging and (ii) the membrane-bound reporter protein can be expressed in diverse cellular context driven by different promoters (constitutive, chemically inducible or inducible by biological processes). Furthermore, iii) the reporter protein itself is modular in a way that it features further to mandatory protein domains (secretory signal peptide, Anticalin, membrane anchor) also optional protein domains such as the extracellular V5-epitope tag and the intracellular fluorescent proteins (FIG. 2). The gene transfer of the reporter gene of the invention into the cell of interest has been established with three different methods: (i) targeted CRISPR/Cas9-mediated genome integration after plasmid transfection or nucleofection and subsequent selection using appropriate antibiotics, (ii) lentiviral or retroviral gene transfer with stable random integration into the genome and (iii) adeno-associated viral vectors that lead to transient gene expression without genomic integration.

TABLE 1
Prior art Anticalins that bind small-molecule ligands

| | CL31d | D6.4(Q77E) | | | |
|---|---|---|---|---|---|
| | (also | (also | | | |
| | abbreviated | abbreviated | | | |
| | herein as | herein | | | |
| Anticalin | DTPA) | as Colchi) | M2 | DigA16(H86N) | FluA(R95K/A45I/S114R) |
| Ligand | Bn-CHX-A″- | Colchicine | Petrobactin | Dig(it)oxigenin | Fluorescein |
| | DTPA•Me | | | | |
| Literature | (Kim et al., 2009) | (Barkovskiy et al., 2018) | (Dauner et al., 2018) | (Schlehuber et al., 2000) | (Vopel et al., 2005) |
| | (Eggenstein | | | | |
| | et al., 2013) | | | | |
| Patent | WO2009156456 | WO2011069992 | WO2011069992 | WO2000075308 | WO1999016873 |
| Applicatio | | | | | |
| No. | | | | | |
| Scaffold | human Lcn2 | human Lcn2 | human Lcn2 | bilin-binding | bilin-binding |
| used | | | | protein | protein |
| Affinity | ~500 pM | ~450 pM | ~20 pM for Fe^{III} | 350 pM | ~1 nM |
| (K_{D}) | | | ~50 PM for Ga^{III} | | |
| Radioligand | CHX-A″- | ¹⁸F-Py-PEG(4)-Colchicine | Petrobactin•⁶⁸Ga^{III} | ¹⁸F-Py-PEG(4)-Digoxigenin | ¹⁸F-Py-PEG(4)-Fluorescein |
| | DTPA•¹⁵²Tb | | | | |
| | (or dimers thereof) | | | | |
| | or ¹⁸F- DTPA•Tb | | | | |
| Advantages | Radiometal- | Perfectly suitable ¹⁸F- | Gallium-68 labeled | No more patent | Patent protection |
| | charged | radiolabeling due to the | radioligand allows easy | protection by | by Pieris Pharma. |
| | CHX″- | lack of acidic protons | radiolabeling | Pieris Pharma. | |
| | DTPA can be used | | | will expire soon | |

Anticalins binding suitable small-molecule radioligands with high affinity (see Table 1) need to be expressed in sufficient density on the surface of the engineered/transformed cell in order to allow complex formation between the Anticalin and its cognate radioligand in reasonable amounts. This design is somewhat similar to a membrane-based reporter gene that binds a DOTA⋅Me complex (Wei et al., 2008, Krebs et al., 2018). This reporter gene was made of the following protein moieties: (i) a signal peptide, (ii) a murine antibody scFv fragment (2D12.5/G54C), (iii) a transmembrane domain from CD4 and (iv) a P2A-separated green fluorescent protein (GFP) (Krebs et al., 2018). Disadvantages of this design, which is based on a single chain variable fragment (scFv) binding protein include: 1) oligomerisation of the antibody fragments (Hudson and Kortt, 1999), 2) low surface expression, 3) murine origin and, hence, potential immunogenicity in humans, 4) the cross-reactive radioligand lanthanoid(S)-2-(4-acrylamidobenzyl)-DOTA (AABD) which, as acrylamide derivative, unspecifically binds also to biological structures other than the DAbR1 reporter protein. In contrast, advantages of the lipocalin-based reporter proteins according to the invention include, but are not limited to: 1) human origin of the protein scaffold, 2) smaller size of the reporter protein, 3) single-chain protein architecture by nature, 4) high stability of the folded protein and 5) the absence of oligomerisation tendency. While antibodies exist in nature both in soluble and in a membrane-anchored form, lipocalins are exclusively found as soluble secretory proteins in mammals. ApoD may be seen as a rare exception as it is found associated with high density lipoprotein particles; however, this lipocalin does not carry a transmembrane domain or GPI anchor (Schiefner and Skerra, 2015).

In one embodiment, the reporter protein of the invention (FIG. 2) is composed of (i) the natural Lcn2 signal peptide [SEQ ID NO:1/2], (ii) a small-molecule-binding Anticalin (see Table 1 and [SEQ ID NO:3/4 or 5/6]), (iii) e.g. the V5-epitope-tag [SEQ ID NO:7/8], (iv) the transmembrane domain of human CD4 [SEQ ID NO:9/10] or CD28 [SEQ ID NO:11/12] and/or a GPI-anchor sequence [SEQ ID NO:13/14 or 15/16] and, optionally, (v) a fluorescent protein (such as mRuby3 [SEQ ID NO:17/18], miRFP703 and miRFP720 [SEQ ID NO:19/20]). In one embodiment, the fluorescent protein is separated from the reporter protein using a 2A self-cleaving peptide sequence. The reporter protein without a fluorescent protein (which may be suitable for method development and preclinical studies) only contains 259 amino acid residues (FIG. 3), which offers the advantage of a minimal genetic design with 777 base pairs (bp) [SEQ ID NO:23/24], thus allowing incorporation together with other functional elements into viral vectors with limited packaging capacity, such as the AAV (with a maximal packaging size only 4700 bp).

Various different expression vectors for the different reporter proteins have been constructed and tested (FIG. 3). These vectors comprise variants of the reporter protein for different forms of gene transfer, such as transfection (FIG. 3A-G), lentiviral or retroviral gene transfer (FIG. 3 H-J) or recombinant AAV gene transfer (FIG. 3 K,L).

Some applications as well as the beneficial properties of the reporter proteins according to this invention will be described and illustrated in the following Examples.

### EXAMPLE 2

### Demonstration of High Expression Levels, Correct Intracellular Transport to the Cell Membrane and Absence of Proteolytic Cleavage

High surface expression of exemplary constructs on transfected cells was verified by flow cytofluorometry (FIG. 4) and western blot analysis (FIG. 5). Flow cytometry analysis was performed and confirmed a much higher surface expression of the invention compared to the Sodium-Iodide Symporter (NIS) which has been often proposed as a reporter protein (Ravera et al., 2017). The surface expression level of the reporter protein of the invention was higher compared to the NIS-mRuby3 but lower than the fluorescent protein mRuby3 expressed as a soluble protein in the cytoplasm (FIG. 4A). Surprisingly, those reporter proteins lacking a fluorescent protein revealed 5-10 fold higher expression of the reporter protein on the cell surface, thus omitting the fluorescent protein for a given application of the reporter protein can be used to increase the expression level of the reporter protein and thus the signal that can be generated (FIG. 4B). Furthermore, the integrity of the reporter protein within T-cells and the absence of shedding of the extracellular domain comprising the Anticalin has been confirmed by western blot analysis of different stably transduced Jurkat cell lines using the V5-tag (FIG. 5).

Finally, the sub-cellular distribution pattern of the fluorescent protein mRuby3 as part of the longer reporter gene construct was investigated in transgenic HEK-(FIG. 6A) and PC3-cell (FIG. 6A) lines by fluorescence microscopy. The V5-tag as well as the intrinsic fluorescence of the mRuby3 were both detected at the cellular membrane of the cells, confirming the efficient secretion and membrane incorporation of the reporter proteins. This membrane localization was independent of the used lipocalin-derived binding proteins (Bn-CHX-A″-DTPA- or Colchicine-binding) and also independent of whether a fluorescent protein was incorporated into the design (FIG. 5).

### EXAMPLE 3

### Exploiting the V5-Epitope Tag for MACS and IHC

The incorporation of an epitope-tag into the reporter protein enables the use of antibodies against this tag for the detection of the protein on the cell surface (e.g. flow cytometry, masscytometry or MACS) or in an isolated form (e.g. western blot). For these applications, the V5-epitope tag (Southern et al., 1991, Dunn et al., 1999) was selected because of its high affinity (K_{D}=24 pM for SV5-PK1), the strong denaturing conditions that are required to break this interaction (9 M urea and 1% Tween-20) as well as the hydrophilic and only slightly charged amino acid sequence (GKPIPNPLLGLDST, see SEQ ID NO:7/8) of the tag (Dunn et al., 1999).

The possibility to bind an accessible epitope on the cell surface with a high affine antibody was used for magnetic-activated cell sorting (FIG. 7). For this technique, the transgenic cells were stained with the SV5-PK1 antibody and subsequently incubated with an antibody against the murine Fc-domain that was conjugated with (super)paramagnetic beads that can be magnetized in a magnetic field. The decoration of the cell surface of the transduced cells was then utilized in a second step to retain the transgenic cells within a magnetic field, while at the same time the cells that are not linked to magnetic particles were washed from the column by the gravity flow of the MACS buffer. Using this method, a purity of ˜99% transgenic cells (FIG. 7) could be achieved which represents an interesting additional benefit of the invention, given the fact that MACS is often used in the production and purification process of patient-derived, autologous CAR T-cells.

Furthermore, the presence of the V5-epitope tag on the cell surface was used to identify the transgenic cells on a cellular level in a histological context (FIG. 8). For this application, the SV5-PK1 antibody was used for immunohistochemical staining of tissue sections which allowed the easy and clear identification of transgenic cells with a high contrast (FIG. 8) which is a big advantage compared to the IHC staining of a cell type of interest with antibodies against naturally occurring antigens. In the Jurkat xenograft tumor model that was investigated, it was possible to clearly distinguish between DTPA-R positive T-cells and murine cells in which the tumor cells had invaded (FIG. 8B) while this is not possible in consecutive HE stained tissue sections (FIG. 8C). This was also true for tissue that was mainly made up of tumor cells with a strong DAB-staining, where the non-stained blood vessel and the nerve fiber in the left part of the tissue section are clearly visible and distinguishable from the labelled cells (FIG. 8D).

### EXAMPLE 4

### The Reporter Protein is well Tolerated in Cells

When the reporter gene system of the invention is used for PET-detection of a cell population of interest, such as infused CAR T-cells or cells transduced with an AAV viral vector, an important prerequisite for the application of the reporter protein is the absence of negative impact on the therapeutic function of this cell population.

In order to check some of the major aspects that may be impaired, stable Jurkat cell lines with different reporter proteins were established and subsequently the doubling time of these cell lines (FIG. 9A) as well as the reporter protein surface expression levels of these cell lines (FIG. 9B) were determined.

The determination of the doubling time for these Jurkat cell lines using the CFSE cell proliferation assay confirmed the absence of a negative effect of the expression of the reporter gene on the cell division rate, as there were no significant differences between the doubling times determined for these cell lines. Although, the unspecific activation of the T-cell using PMA/ionomycin slightly increased the doubling time of all cell lines, there was no detectable difference between all evaluated reporter gene constructs and the untransduced control cells (FIG. 9A).

The determination of the total cell surface expression of the reporter gene was conducted with anti-V5 antibody that had been labeled with a fluorescent dye for which a kit with calibration beads was available. The results for the total number of receptors on the cell surface reflected the results earlier determined for different reporter gene constructs when quantifying the intrinsic fluorescence of the fluorescent protein (FIG. 4). The highest expression was measured for Jurkat transduced with DTPA-R with a mean of 870.000 copies on the cell surface (FIG. 9B). For all the cell lines measure the activation with PMA/ionomycin increased the surface expression of the reporter protein slightly, as T-cell activation is known to increase size and metabolic activity. For the Jurkat DTPA-R cell line, the surface expression of 870.000 copies was increased upon T-cell activation by ˜10% to 960.000 (FIG. 9B).

Thus, it is not only confirmed that the expression of different reporter proteins of the invention does not change the proliferation rate, but also that major events in the physiology of the T-cell only have a minor effect on the surface expression of the reporter gene.

Furthermore, anti-CD19 CAR T-cells created from peripheral blood mononuclear cell (PBMC) of a healthy donor were evaluated for their potential to kill CD19-positive target cells. The cellular toxicity to the target cells was measured by a radioactive Chrome-51 release assay at a 4 h end point (FIG. 10). The assay was conducted with B-cell precursor leukemia cell line NALM-6 (FIG. 10A) and the Burkitt lymphoma cell line Raji (FIG. 10B). With both target cell lines it was confirmed that the PBMCs transduced with CAR/DTPA-R showed equal killing efficacy compared to the PBMCs transduced with CAR/EGFRt, which is a successful CAR construct known to literature and serves as a reference standard (Wang et al., 2011, Paszkiewicz et al., 2016).

This result was furthermore confirmed by a FACS based killing assay in which the successful killing of GFP-expressing NALM-6 target cells was confirmed by both CAR T-cells, CAR/EGFRt and CAR/DTPA-R, respectively (FIG. 10 C-F).

### EXAMPLE 5

### Design of Radiopharmaceuticals for the Use with the Reporter Proteins of the Invention

It is crucial for the development of both a preclinically and a clinically useful reporter system to select the best affinity pair composed of a genetically encodable binding protein (here an Anticalin) and its ligand. Relevant criteria for this choice include: (i) the affinity of the binding protein to the small molecule ligand (see Table 1), (ii) the availability of different radiolabeling strategies to generate radioligands with different properties (FIG. 13), (iii) the in vivo distribution, clearance and metabolization properties of the radioligand, (iv) the pharmacological knowledge about the ligand and its previous use in medicine, (v) a high molar/specific activity and (vi) technical aspects of the radioisotope and the radiosynthesis itself.

Given these criteria, different ligands were synthesized (FIG. 11-13), their chemical properties were tested and their binding to transformed cells, followed by investigation of their biodistribution in vivo in mice. The result was that the incorporation of one or two D-Glu amino acid residues into the radioligand for use together with the Colchi-R increased the hydrophilicity drastically, as expected (FIG. 11). This increase in hydrophilicity is an intended characteristic of radiopharmaceuticals as it facilitates the clearance of the radioligand from the tissue and, thus, improves the signal to background ratio, which is necessary to detect even weak signals in PET, which are expected from small cell populations. While the ¹⁸F-Py-PEG(4)-colchicine radioligand had a LogP value of 0.89 and, thus, only 11.4% was found in the PBS phase, the incorporation of one D-Glu residue improved the LogP to a more hydrophilic value of −1.92 (98.8% in PBS phase) and a second D-Glu residue even brought the LogP to −2.97 (99.9% in the PBS phase). The D-Glu residues were chosen over the proteinogenic L-Glu residues as they are known to limit the susceptibility of the resulting radioligand for proteolytic cleavage and, thus, improve the stability of the ligand in vivo (Grishin et al., 2020). Furthermore, a binding motif for a serum or plasma protein known in the art may be included into the linker, as it has been shown to modify the tissue distribution profile and extend the plasma half-life of small molecule ligands (Benesova et al., 2018). Such binding motifs could for example be selected from known albumin binding domains (Dumelin et al., 2008). The fluorination was accomplished by the incorporation of a nicotinic acid building block that featured a trimethyl-ammonium (TMA) leaving group that allowed the one-step radiofluorination of the precursor molecule according to the “minimalist approach” (Richarz et al., 2014). The advantage of this new method lies in the robust fluorination reaction that is based on a simple nucleophilic substitution reaction in which the positively charged TMA leaving group is exchanged for the radioactive Fluorid-18 (FIG. 12A). Furthermore, the positive charge within the leaving group allows the separation of the non-radioactive precursor and the radioactive radioligand by cartridges or chromatography in order to obtain an optimal specific (molar) activity of the radioligand. The different steps in the radiolabeling and purification process are depicted and illustrate the fact that cartridge purification with a QMA and C18 cartridge is sufficient to purify the product of the radiolabeling reaction to an acceptable purity, while this can even be improved by a final preparative HPLC run (FIG. 12B).

Finally, the radioligands for the Colchi-R were injected into nude mice and a dynamic PET-scan was recorded. While the biodistribution of the ¹⁸F-Py-PEG(4)-colchicine radioligand is dominated by its lipophilic nature (positive LogP value) and, thus, is only eliminated insufficiently from the organism (FIG. 12C), the incorporation of the D-Glu residues improves hydrophilicity and, thus, improves the clearance from the tissue via the kidneys (FIG. 15A). Nevertheless, the compound is not sufficiently hydrophilic to be exclusively cleared via the kidneys.

Besides the radioligands for the Colchi-R (FIG. 11-12), also various radioligand for the DTPA-R were designed and synthesized (FIG. 13). These radioligands are all based on the binding motive that is tightly bound by the respective Anticalin, the NH₂-Bn-CHX-A″-DTPA (FIG. 13A). Based on this ligand moiety, which can be used to chelate radio-metals suitable for diagnostic imaging or therapy, novel precursors for radiofluorination (FIG. 13BC) were constructed. Finally, also bivalent DTPA radioligands were constructed that feature two interconnected ligand moieties that can simultaneously bind to two different Anticalins on the cell surface and, thus, improve the overall affinity of the radioligand in vitro and in vivo (FIG. 13DE). These radioligand feature an improved affinity and are able to also deliver radionuclides for therapeutic purposes, such as Y-90, Tb-149, Tb-161, Bi-213 or a combination of such a therapeutic radioisotope together with a cold metal ion.

### EXAMPLE 6

### Specific Binding of the Radioligand to the Reporter Protein In Vitro and In Vivo

For the sensitive and specific detection of cells that are labeled with the reporter gene of the invention, the binding between the Anticalin binding protein and the radioligand is of utmost importance. For this reason, the binding of different radioligands to cells that were stably modified with the reporter genes was studies in vitro (FIG. 14). At first, the binding of the ligand domain for the DTPA-R (NH₂-CHX-A″-DTPA) to Jurkat cells expressing the reporter protein DTPA-V5-TMD-mRuby3 was assessed. For this binding study, the radioactive Yttrium isotope Y-90 was chelated by the ligand domain and a fixed amount of the radioligand was applied to the cells that had been previously incubated with a dilution series of the same chelator molecule that had been charged with the cold Yttrium isotope Y-89. Curve fitting of the obtained binding curve resulted in a IC₅₀-value of 1.4 nM, demonstrating the high-affine binding of the radioligand to the Anticalin on the cell surface (FIG. 14A). In order to test the specificity of the reporter protein—Colchicine radioligand interaction, the binding was blocked by a high molar excess of the ligand domain (here 200 μM colchicine) prior to incubation with the radioligand (FIG. 14B). Independent of the cell type in which the Colchi-R has been expressed (Jurkat or PC3), there was high accumulation of the radioligand in the cell fraction after washing them twice, while there was no signal detectable for the DTPA-R expressing cells. Comparable low levels of bound radioligand were measured for the cells that have been blocked with the colchicine prior to the application of the radioligand, again proving the specificity of the radioligand binding. Finally, the orthogonality of the DTPA-R and the Colchi-R have been studied in a single cell binding experiment (FIG. 14C). To this end, Jurkat cells expressing the respective reporter genes or untransduced Jurkat cells were incubated with the two radioligands, each. The result clearly shows that the DTPA radioligand only binds to the DTPA-R cells and the Colchi radioligand is specific for the Colchi-R modified cells. At the same time, there was no detectable binding of any radioligand to the cells of the second Anticalin-ligand pair or to the wild type Jurkat cells (FIG. 14C). Taken together, these experiments clearly demonstrate the high affinity of the membrane-bound Anticalin for the respective radioligand in an affinity-range comparable to the affinity determined for the recombinant Anticalin. Furthermore, the binding is specific to the respective reporter protein and there is no unspecific binding detectable.

The ability to use the reportergene system for in vivo PET-imaging was assessed (FIG. 15-17). For this experiment xenograft tumors were inoculated in nude mice and the mice where then used for the PET-imaging experiments. The intravenous injection of the radioligand ¹⁸F-Py-D-Glu-D-Glu-PEG(4)-Colchicine resulted in a systemic distribution of the radioligand and a rapid clearance via the hepato-biliary system into the colon (FIG. 15A). Within the xenograft tumor that had been grown from Colchi-R positive cells, there was a clear and strong accumulation of the radioligand. In contrast, the xenograft tumor that had been grown from DTPA-R expressing cells showed no accumulation of ¹⁸F-Colchicine above the level of the surrounding tissue. Upon an injection of the radioligand the PC3 xenograft showed an accumulation of the radioligand. This experiment clearly demonstrates the ability of the invention to allow the specific and sensitive detection of transgenic cells in vivo by means of Colchi-R PET-imaging. Furthermore, the radioligand of DTPA-R was tested in the same animal model comprising two PC3 xenograft tumors expressing both reporter proteins. The ¹⁸F-DTPA radioligand ¹⁸F-Py-D-Glu-D-Glu-PEG(4)-DTPA⋅Tb was synthesized and injected into a tumor-bearing nude mouse and after 60 min a static PET-scan was recorded (FIG. 15B). Even the very small PC3 DTPA-R xenograft tumor of only 11 mm³ was clearly visible in the MIP of the PET-scan as well as in the axial sections (shown below). Besides the desired signal of the tumor cells that were labeled with the DTPA-R reporter protein, only minor elimination related signal could be detected. These signals in gall bladder, kidneys, ureter and urine bladder are located in well defined tissues and organs of the animal and, thus, allow the differentiation between reporter protein related and elimination related PET-signals. Using later image time frames for the PET-scan, it will obviously be possible to allow the ¹⁸F-DTPA to even further eliminate from the organism and to obtain an even cleaner background for the detection of DTPA-R tagged cells. Besides the exquisite specificity of the reporter gene PET-imaging system of the invention also the sensitivity of the system is of importance. Besides the clear detection of very small xenograft tumors (FIG. 15A), also the detection limit was determined with Jurkat cells expression the DTPA-R. These cells were incubated with an excess of the radioligand and were then washed twice. Subsequently, a dilution series in PCR tubes was prepared and scanned for 1 h in the Mediso nano-PET/MR scanner (FIG. 15 C). Here the detection limit was at ˜5.000 cells that showed enough signal to be discriminated from the background.

To further analyze the dynamics of the ¹⁸F-DTPA⋅Tb radioligand in vivo, a dynamic PET-scan was conducted for 90 min post injection. An equal distribution of the radioligand in the whole animal can be seen in the first 5 minutes, which is then followed by a rapid elimination of the radioligand via the kidneys. Already after 30 minutes, a clear contrast is visible that allows the identification of the tumor that was DTPA-R modified, while the Colchi-R labeled tumor xenograft remained invisible. Again, elimination related PET-signals were visible and somewhat more pronounced because the lack of physical activity of the mouse during anesthesia led to a slower clearance of the radioligand. At the same time, the signal of the radioligand within the tumor remained constant over the whole 90 minutes of the dynamic PET-scan, which is caused by the high affinity of the Anticalin for the DTPA ligand-moiety that efficiently limits the dissociation and subsequent elimination of the radioligand. In addition, it must be pointed out, that the radioligand only accumulated in the vital border regions of the xenograft tumor, while the core region containing secrete and necrotic cells showed no PET-signal above background level (FIG. 16C-D). This clearly proves that the signal obtained from a ¹⁸F-DTPA scan is limited to living cells and, thus, no false-positive signal from dead cells is measured. Finally, it was confirmed that the reporter gene can also be delivered to host cells via a genetic vector. For this purpose plasmids were constructed featuring inverted terminal repeats (ITRs) of the adeno-associated virus serotype 2 and respective expression cassette for two reporter proteins of the invention (FIG. 17A). These plasmids where then subsequently used to produce AAV viral vector of the serotype 9 encoding the reporter proteins. In an in vitro experiment different titers of these viral preparations were used to transduce HEK cells and resulting fluorescence intensities as well as the fraction of the DTPA-R-miRFP720 or Colchi-R-miRFP720 positive cells was determined (FIG. 17B). These viral vectors were also used in in vivo experiments in nude mice, which received an i.v. injection of the viral vectors. The expression of the Colchi-R after viral transduction events in different cells within the myocardial muscle of the mouse was detected via IHC staining. For the DTPA-R, also a PET-scan was used to detect viral transduction events in a quantitative and sensitive manner. This PET-scan resulted in the detection of a pronounced signal within the heart of the mouse but also muscles in the neck and dorsal region where clearly visible. Thus, it can be claimed that the reporter proteins of the invention are suitable for the quantification of AAV transduction and transgene expression in a longitudinal in vivo study.

### EXAMPLE 7

### Advantage of Using Anticalins Instead of Other Binding Proteins

As explained herein above in Example 1, a murine antibody scFv fragment (2D12.5/G54C) was previously employed as a reporter protein for PET imaging (Krebs et al., 2018). This scFv fragment, binds DOTA:metal complexes and was derived from the murine monoclonal antibody 2D12.5/G54C—It was used to construct the synthetic reporter gene DabR1 (DOTA Antibody Reporter 1) (Krebs et al., 2018). Based on the Antibody 2D12.5, also scFv fragments with improved affinity were developed and proposed as binding proteins which can be used as part of a membrane-anchored reporter protein (improved version of DAbR1). The corresponding muC825 scFv as well as its humanized version, huC825 scFv, were described for example in EP3256164B1 and WO 2019/060750A2. To best knowledge of the inventors, the scFv huC825 represents the most advanced and most appropriate candidate of an antibody-derived binding protein for the construction of a PET reporter gene according to the art (Dacek et al., 2021)(Krebs et al., 2018). Due to the increased affinity of C825 for DOTA:metal complexes, the complicated formation of a covalent bond between the engineered Cys residue G54C and the acrylamidobenzyl group as part of the radioligand (Krebs et al., 2018) was no longer necessary. The free Cys residue in the oxidizing extracellular milieu is prone to chemical modification while the chemical cross-reactivity of the acrylamidobenzyl group leads to undesired background signals. Apart from that, the humanization can be expected to reduce the potential immunogenicity of the murine protein in immunocompetent patients, which is of high importance for clinical translation (Dacek et al., 2021).

Both the murine and the humanized scFv C825 were compared with the Anticalin-based reporter protein according to this invention side by side. To this end, three membrane-anchored reporter proteins were constructed which only differ in the binding protein domain specific for the radioactive chelator:metal complex. These were (a) the DTPA-binding Anticalin CL31d (SEQ ID NO 23/24, FIG. 18A), (b) the murine C825 scFv (SEQ ID NO: 30, FIG. 18B) and (c) the humanized C825 scFv (SEQ ID NO: 31, FIG. 18C).

The amino acid sequences for the murine and human scFv, including the signal peptide (MGWSCIILFLVATATG; SEQ ID NO: 32), were taken from WO 2019/060750A2 and codon-optimized for Homo sapiens. The synthesized genes were cloned using Pacl and Clal restriction sites on pMP71-Lcn2_SignalP-CL31d-V5-V5-CD4TMD, that is the plasm id used for the retroviral transduction with Lcn2_SignalP-CL31d-V5-CD4TMD. Sanger sequencing confirmed correctness of the coding regions and, subsequently, the three plasmids were used for the production of retroviral vectors (Engels et al., 2003), followed by transduction of the human T-cell line Jurkat. The amount of virus was chosen to achieve a low transduction rate (20-33%) in order to avoid events of multiple transduction and genomic integration.

Transduced Jurkat cells were then stained with the AlexaFluor488-conjugated V5-binding IgG antibody SV5-PK1. Surface density of the reporter constructs in the three stably transduced cell lines was analyzed under mutually identical conditions by detection of the V5-tag via flow cytometry in two independent experiments (FIG. 18D-G). The histogram of the fluorescence signals is shown for all three constructs (FIG. 18D), demonstrating a huge difference in the surface expression levels as assessed from the Median Fluorescence Intensity (MFI). Both scFv-based reporter proteins showed only a very low expression (MFI=1'134 and 1'078 for murine and humanized scFv (C825), respectively). In contrast, the Anticalin-based reporter protein exhibited a very high expression level (MFI=6'559 for CL31d). This corresponds to a 5.8/6.1-fold difference in the MFI between the Anticalin-based reporter construct according to this invention and the corresponding constructs employing scFv binding moieties as known in the art.

Furthermore, the MFI values were converted into absolute numbers of detected fluorescent dye molecules (as described in the legend of FIG. 9) to assess the absolute number of receptors per cell (FIG. 18E). While mu_scFv(C825) revealed a median of 31'600 and hu_scFv (C825) revealed a median of 28'600 receptors per cell, the Anticalin-based reporter construct resulted in a median of 260'700 receptors per cell. This corresponds to a 8.2/9.1-fold higher cell surface expression level for the Anticalin-based reporter protein design according to this invention. This strong effect is somewhat reduced after fluorescence activated cell sorting (FACS) of the 10% highest expressing clones (FIG. 18F), after which the muC825 cell line had 292'000 receptors, huC825 expressed 237'000 receptors, while CL31d displaying cells showed an expression of 886'000 receptors per cell (FIG. 18G). While the difference was reduced to a 3.0/3.7-fold higher expression for the Anticalin-based design after this more stringent selection process, the use of the Anticalin-based design still offers a significant advantage over reporter gene systems known in the art. As all three mRNAs were expressed from the same promoter, with the same 5′ UTR, and the three reporter constructs that were compared here feature the same membrane anchor domain, the difference in expression levels must be attributed to the inferior properties of the antibody-derived scFvs. The use of the (Lcn2) Anticalin scaffold, which naturally constitutes a single polypeptide chain with stable fold that is not prone to aggregation, offers a clear improvement over scFv fragments, for which poor folding efficiency and a tendency towards oligomer formation and aggregation is known in the art. In fact, the ˜9-fold higher expression level on the cell surface, as demonstrated in this example, should lead to the binding of a ˜9-fold larger number of radioligands and allow for the much more sensitive detection of transduced or transfected cells in PET imaging studies.

### REFERENCES

BAJAR, B. T., WANG, E. S., LAM, A. J., KIM, B. B., JACOBS, C. L., HOWE, E. S., DAVIDSON, M. W., LIN, M. Z. & CHU, J. 2016. Improving brightness and photostability of green and red fluorescent proteins for live cell imaging and FRET reporting. Sci Rep, 6, 20889.
BARKOVSKIY, M., ILYUKHINA, E., DAUNER, M., EICHINGER, A. & SKERRA, A. 2018. An engineered lipocalin that tightly complexes the plant poison colchicine for use as antidote as well as bioanalytical applications. Biol Chem.
BENESOVA, M., UMBRICHT, C. A., SCHIBLI, R. & MULLER, C. 2018. Albumin-Binding PSMA Ligands: Optimization of the Tissue Distribution Profile. Mol Pharm, 15, 934-946.
BRAASCH, D. A. & COREY, D. R. 2001. Locked nucleic acid (LNA): fine-tuning the recognition of DNA and RNA. Chem Biol, 8, 1-7.
BRADER, P., SERGANOVA, I. & BLASBERG, R. G. 2013. Noninvasive molecular imaging using reporter genes. J Nucl Med, 54, 167-72.
DACEK, M. M., VEACH, D. R., CHEAL, S. M., CARTER, L. M., MCDEVITT, M. R., PUNZALAN, B., BURNES VARGAS, D., KUBIK, T. Z., MONETTE, S., SANTICH, B. H., YANG, G., OUERFELLI, O., KESNER, A. L., CHEUNG, N. V., SCHEINBERG, D. A., LARSON, S. M. & KREBS, S. 2021. Engineered Cells as a Test Platform for Radiohaptens in Pretargeted Imaging and Radioimmunotherapy Applications. Bioconjug Chem, 32, 649-654.
DAUNER, M., EICHINGER, A., LUCKING, G., SCHERER, S. & SKERRA, A. 2018. Reprogramming Human Siderocalin To Neutralize Petrobactin, the Essential Iron Scavenger of Anthrax Bacillus. Angew Chem Int Ed Engl, 57, 14619-14623.
DERENZO, C. & GOTTSCHALK, S. 2019. Genetic Modification Strategies to Enhance CAR T Cell Persistence for Patients With Solid Tumors. Front Immunol, 10, 218.
DUMELIN, C. E., TRUSSEL, S., BULLER, F., TRACHSEL, E., BOOTZ, F., ZHANG, Y., MANNOCCI, L., BECK, S. C., DRUMEA-MIRANCEA, M., SEELIGER, M. W., BALTES, C., MUGGLER, T., KRANZ, F., RUDIN, M., MELKKO, S., SCHEUERMANN, J. & NERI, D. 2008. A portable albumin binder from a DNA-encoded chemical library. Angew Chem Int Ed Engl, 47, 3196-201.
DUNN, C., O'DOWD, A. & RANDALL, R. E. 1999. Fine mapping of the binding sites of monoclonal antibodies raised against the Pk tag. J Immunol Methods, 224, 141-50.
EGGENSTEIN, E., EICHINGER, A., KIM, H. J. & SKERRA, A. 2013. Structure-guided engineering of Anticalins with improved binding behavior and biochemical characteristics for application in radio-immuno imaging and/or therapy. J Struct Biol.
ENGELS, B., CAM, H., SCHULER, T., INDRACCOLO, S., GLADOW, M., BAUM, C., BLANKENSTEIN, T. & UCKERT, W. 2003. Retroviral vectors for high-level transgene expression in T lymphocytes. Hum Gene Ther, 14, 1155-68.
GEBAUER, M. & SKERRA, A. 2012. Anticalins small engineered binding proteins based on the lipocalin scaffold. Methods Enzymol, 503, 157-88.
GHIM, C. M., LEE, S. K., TAKAYAMA, S. & MITCHELL, R. J. 2010. The art of reporter proteins in science: past, present and future applications. BMB Rep, 43, 451-60.
GRISHIN, D., ZHDANOV, D., POKROVSKAYA, M. & SOKOLOV, N. 2020. D-amino acids in nature, agriculture and biomedicine. All Life, 13, 11-22.
GRUTZKAU, A. & RADBRUCH, A. 2010. Small but mighty: how the MACS-technology based on nanosized superparamagnetic particles has helped to analyze the immune system within the last 20 years. Cytometry A, 77, 643-7.
HUDSON, P. J. & KORTT, A. A. 1999. High avidity scFv multimers; diabodies and triabodies. J Immunol Methods, 231, 177-89.
KIM, H. J., EICHINGER, A. & SKERRA, A. 2009. High-affinity recognition of lanthanide(III) chelate complexes by a reprogrammed human lipocalin 2. J Am Chem Soc, 131, 3565-76.
KIM, J. H., LEE, S. R., LI, L. H., PARK, H. J., PARK, J. H., LEE, K. Y., KIM, M. K., SHIN, B. A. & CHOI, S. Y. 2011. High cleavage efficiency of a 2A peptide derived from porcine teschovirus-1 in human cell lines, zebrafish and mice. PLoS One, 6, e18556.
KREBS, S., AHAD, A., CARTER, L., EYQUEM, J., BRAND, C., BELL, M., PONOMAREV, V., REINER, T., MEARES, C. F., GOTTSCHALK, S., SADELAIN, M., LARSON, S. M. & WEBER, W. A. 2018. Antibody with infinite affinity for in vivo tracking of genetically engineered lymphocytes. J Nucl Med, 59, 1894-1900.
KUMAGAI, A., ANDO, R., MIYATAKE, H., GREIMEL, P., KOBAYASHI, T., HIRABAYASHI, Y., SHIMOGORI, T. & MIYAWAKI, A. 2013. A bilirubin-inducible fluorescent protein from eel muscle. Cell, 153, 1602-11.
LABANIEH, L., MAJZNER, R. G. & MACKALL, C. L. 2018. Programming CAR-T cells to kill cancer. Nat Biomed Eng, 2, 377-391.
LI, C. & SAMULSKI, R. J. 2020. Engineering adeno-associated virus vectors for gene therapy. Nat Rev Genet, 21, 255-272.
LIU Z, CHEN O, WALL J B J, ZHENG M, ZHOU Y, WANG L, VASEGHI H R, QIAN L, LIU J. 2017. Systematic comparison of 2A peptides for cloning multi-genes in a polycistronic vector. Sci Rep, 7, 2193.
LUNDSTROM, K. 2018. Viral Vectors in Gene Therapy. Diseases, 6.
MILIOTOU, A. N. & PAPADOPOULOU, L. C. 2018. CAR T-cell Therapy: A New Era in Cancer Immunotherapy. Curr Pharm Biotechnol, 19, 5-18.
MILTENYI, S. & SCHMITZ, J. 2000. High Gradient Magnetic Cell Sorting. In: RADBRUCH, A. (ed.) Flow Cytometry and Cell Sorting. Berlin, Heidelberg: Springer Berlin Heidelberg.
OWENS, G. C., CHAPPELL, S. A., MAURO, V. P. & EDELMAN, G. M. 2001. Identification of two short internal ribosome entry sites selected from libraries of random oligonucleotides. Proc Natl Acad Sci USA, 98, 1471-6.
PASZKIEWICZ, P. J., FRASSLE, S. P., SRIVASTAVA, S., SOMMERMEYER, D., HUDECEK,

M., DREXLER, I., SADELAIN, M., LIU, L., JENSEN, M. C., RIDDELL, S. R. & BUSCH, D. H. 2016. Targeted antibody-mediated depletion of murine CD19 CAR T cells permanently reverses B cell aplasia. J Clin Invest, 126, 4262-4272.
PAULICK, M. G. & BERTOZZI, C. R. 2008. The glycosylphosphatidylinositol anchor: a complex membrane-anchoring structure for proteins. Biochemistry, 47, 6991-7000.
PRICE, E. W., EDWARDS, K. J., CARNAZZA, K. E., CARLIN, S. D., ZEGLIS, B. M., ADAM, M. J., ORVIG, C. & LEWIS, J. S. 2016. A comparative evaluation of the chelators H4octapa and CHX-A″-DTPA with the therapeutic radiometal (90)Y. Nucl Med Biol, 43, 566-576.
PRICE, E. W. & ORVIG, C. 2014. Matching chelators to radiometals for radiopharmaceuticals. Chem Soc Rev, 43, 260-90.
RAVERA, S., REYNA-NEYRA, A., FERRANDINO, G., AMZEL, L. M. & CARRASCO, N. 2017. The Sodium/Iodide Symporter (NIS): Molecular Physiology and Preclinical and Clinical Applications. Annu Rev Physiol, 79, 261-289.
RICHARZ, R., KRAPF, P., ZARRAD, F., URUSOVA, E. A., NEUMAIER, B. & ZLATOPOLSKIY, B. D. 2014. Neither azeotropic drying, nor base nor other additives: a minimalist approach to (18)F-labeling. Org Biomol Chem, 12, 8094-9.
RICHTER, A., EGGENSTEIN, E. & SKERRA, A. 2014. Anticalins: exploiting a non-Ig scaffold with hypervariable loops for the engineering of binding proteins. FEBS Lett, 588, 213-8.
SCHIEFNER, A. & SKERRA, A. 2015. The menagerie of human lipocalins: a natural protein scaffold for molecular recognition of physiological compounds. Acc Chem Res, 48, 976-85.
SCHLEHUBER, S., BESTE, G. & SKERRA, A. 2000. A novel type of receptor protein, based on the lipocalin scaffold, with specificity for digoxigenin. J Mol Biol, 297, 1105-20.
SERGANOVA, I. & BLASBERG, R. 2005. Reporter gene imaging: potential impact on therapy. Nucl Med Biol, 32, 763-80.
SERGANOVA, I., MAYER-KUKUCK, P., HUANG, R. & BLASBERG, R. 2008. Molecular imaging: reporter gene imaging. Handb Exp Pharmacol, 167-223.
SHAH, N. N. & FRY, T. J. 2019. Mechanisms of resistance to CAR T cell therapy. Nat Rev Clin Oncol, 16, 372-385.
SHCHERBAKOVA, D. M., BALOBAN, M., EMELYANOV, A. V., BRENOWITZ, M., GUO, P. & VERKHUSHA, V. V. 2016. Bright monomeric near-infrared fluorescent proteins as tags and biosensors for multiscale imaging. Nat Commun, 7, 12405.
SHCHERBAKOVA, D. M., COX CAMMER, N., HUISMAN, T. M., VERKHUSHA, V. V. & HODGSON, L. 2018. Direct multiplex imaging and optogenetics of Rho GTPases enabled by near-infrared FRET. Nat Chem Biol, 14, 591-600.
SOUTHERN, J. A., YOUNG, D. F., HEANEY, F., BAUMGARTNER, W. K. & RANDALL, R. E. 1991. Identification of an epitope on the P and V proteins of simian virus 5 that distinguishes between two isolates with different biological characteristics. J Gen Virol, 72 (Pt 7), 1551-7.
VARGAS, J. E., CHICAYBAM, L., STEIN, R. T., TANURI, A., DELGADO-CANEDO, A. & BONAMINO, M. H. 2016. Retroviral vectors and transposons for stable gene therapy: advances, current challenges and perspectives. J Transl Med, 14, 288.
VOPEL, S., MUHLBACH, H. & SKERRA, A. 2005. Rational engineering of a fluorescein-binding anticalin for improved ligand affinity. Biol Chem, 386, 1097-104.
WANG, X., CHANG, W. C., WONG, C. W., COLCHER, D., SHERMAN, M., OSTBERG, J. R., FORMAN, S. J., RIDDELL, S. R. & JENSEN, M. C. 2011. A transgene-encoded cell surface polypeptide for selection, in vivo tracking, and ablation of engineered cells. Blood, 118, 1255-63.
WEI, L. H., OLAFSEN, T., RADU, C., HILDEBRANDT, I. J., MCCOY, M. R., PHELPS, M. E., MEARES, C., WU, A. M., CZERNIN, J. & WEBER, W. A. 2008. Engineered antibody fragments with infinite affinity as reporter genes for PET imaging. J Nucl Med, 49, 1828-35.

1. A nucleic acid molecule encoding a fusion protein comprising
(i) a secretory signal peptide;
(ii) a lipocalin-derived binding protein that specifically binds to an exogenous ligand; and
(iii) a glycosylphosphatidylinositol (GPI) anchored and/or transmembrane domain.

2. The nucleic acid molecule of claim 1, wherein the exogenous ligand is linked to a radionuclide.

3. The nucleic acid molecule of claim 1, wherein the encoded fusion protein further comprises a peptide affinity tag.

4. The nucleic acid molecule of claim 1, wherein the encoded fusion protein
further comprises a fluorescent protein.

5. The nucleic acid molecule of claim 1, wherein the exogenous ligand comprises a small molecule, wherein the small molecule is selected from
(i) a chelator,
(ii) an alkaloid,
(iii) an iron-chelating siderophore,
(iv) a plant steroid, and
(v) an organic dye.

6. The nucleic acid molecule of claim 1, wherein the secretory signal peptide is the signal peptide of a lipocalin.

7. The nucleic acid molecule of claim 1, wherein the transmembrane domain is the transmembrane domain of CD4 or CD28.

8. A vector comprising the nucleic acid molecule of claim 1, wherein the vector is a retroviral vector, an adenoviral vector or an adeno-associated vector (AAV).

9. A fusion protein encoded by the nucleic acid molecule of claim 1.

10. A cell transduced or transfected with the nucleic acid molecule of claim 1.

11. The cell of claim 10, wherein the cell is a lymphocyte.

12. The cell of claim 10, wherein the cell further comprises a chimeric antigen receptor or a transgenic T-cell receptor.

13. A kit comprising (i) the nucleic acid molecule of claim 1, and (ii) an exogenous ligand.

14. The cell of claim 10 for use in the treatment of a disease by a cell-based therapy or a gene therapy.

15. An exogenous ligand for use in an in vivo method of diagnosing the efficacy of a cell-based therapy in a subject, wherein the subject has been treated with the cell of claim 10.

16. The nucleic acid molecule of claim 2, wherein the radionuclide is selected from C-11, F-18, Sc-44, Sc-47, Cu-64, Ga-68, Y-86, Y-90, Zr-89, Tc-99m, In-111, I-123, I-124, I-131, Tb-152 and Lu-177.

17. The nucleic acid molecule of claim 3, wherein the peptide affinity tag is selected from a V5-, Strep-II, Flag-, c-myc-, HA-, Spot-, T7-, and NE-epitope tag.

18. The nucleic acid molecule of claim 4, wherein the fluorescent protein is an autofluorescent protein selected from mRuby3, GFP, eGFP, sfGFP, UnaG, miRFP703 and miRFP720.

19. The nucleic acid molecule of claim 1, wherein the encoded fusion protein lacks a fluorescent protein.

20. The nucleic acid molecule of claim 1, wherein the exogenous ligand is a peptide with at least 2 amino acid residues and less than 10 amino acid residues.


SEQUENCE LISTING (29 sequences)

SEQ ID NO: 1 - DNA, 60 bp, Homo sapiens
  Lcn2 signal peptide
atgcctctcggactgctgtggcttggactggctctgctgggagcactgcatgctcaggcc

SEQ ID NO: 2 - protein, 20 aa, Homo sapiens
  Lcn2 signal peptide
MPLGLLWLGLALLGALHAQA

SEQ ID NO: 3 - DNA, 531 bp, Artificial Sequence
  DTPA-binding Anticalin CL31d(A145T)
caggatagcaccagcgatctgattccagctcctccactgagcaaggtgcccctgcagcagaacttccaggacaaccaattccatgggaagtggtatcaagtgggcagagccggaaatgccgctcctcctgaagatcctgaactgctgctgctgacagcccagacctacgagctgaaagaggacaagagctacgacgtgaccgccgtgcggttccggaagaagatgtgcgagtacctgaccatgaccttcgtgcctggatctcagcctggcgagttcaccctgggcaacatcaagaactaccccggcctgaccagctacttcgtcagagtggtgtccaccaactacaaccagcacgccatggtgttcttcaagaaggtccagcagaaccgcgagtacttcagcatctctctgctcggccggaccaaagagctgaccagcgaactgaaagagaacttcatccggttctccaaatctctgggcctgcccgagaatcacatcgtgttccccgtgcctatcgaccagtgcatcgat

SEQ ID NO: 4 - protein, 177 aa, Artificial Sequence
  DTPA-binding Anticalin CL31d(A145T)
QDSTSDLIPAPPLSKVPLQQNFQDNQFHGKWYQVGRAGNAAPPEDPELLLLTAQTYELKEDKSYDVTAVRFRKKMCEYLTMTFVPGSQPGEFTLGNIKNYPGLTSYFVRVVSTNYNQHAMVFFKKVQQNREYFSISLLGRTKELTSELKENFIRFSKSLGLPENHIVFPVPIDQCID

SEQ ID NO: 5 - DNA, 531 bp, Artificial Sequence
  Colchicine-binding Anticalin D6.4(Q77E)
caggatagcaccagcgatctgattccagctcctccactgagcaaggtgcccctgcagcagaacttccaggacaaccaattccatggggagtggtatgtggtaggtgtcgccggcaatggtttcctcagagaagataaagatccgattaaaatggcggccaccatctacgaattaaaagaagacaagtcttataacgtgacctttatgaagtttccaatgaagaaatgcgagtacatgaccgacacgctggttcctggaagtcagcccggcgagtttatccttggcaatatcaagagtgaacctggatacacatcttggctcgtgcgcgtcgtgtcaacaaactataatcagcacgctatggtgttctttaaggctgttcaacagaatcgggaggactttttcattactctttacggaaggacaatagagctgacctccgagctgaaggagaactttattcgattctccaaatctctgggcctgcccgagaatcacatcgtgttccccgtgcctatcgaccagtgcatcgat

SEQ ID NO: 6 - protein, 177 aa, Artificial Sequence
  Colchicine-binding Anticalin D6.4(Q77E)
QDSTSDLIPAPPLSKVPLQQNFQDNQFHGEWYVVGVAGNGFLREDKDPIKMAATIYELKEDKSYNVTFMKFPMKKCEYMTDTLVPGSQPGEFILGNIKSEPGYTSWLVRVVSTNYNQHAMVFFKAVQQNREDFFITLYGRTIELTSELKENFIRFSKSLGLPENHIVFPVPIDQCID

SEQ ID NO: 7 - DNA, 63 bp, Artificial Sequence
  Peptide-linker containing V5-tag
gccagcggcaagcccattcctaatcctctgctgggcctcgattctactggcgcgcctgcttct

SEQ ID NO: 8 - protein, 21 aa, Artificial Sequence
  Peptide-linker containing V5-tag
ASGKPIPNPLLGLDSTGAPAS

SEQ ID NO: 9 - DNA, 105 bp, Artificial Sequence
  CD4 transmembrane domain
cctgttcaacccatggctctgatcgtgcttggcggagttgctggactgctgctgtttatcggcctgggcatcttcttttgcgtgcggtgcagacataggcggaga

SEQ ID NO: 10 - protein, 35 aa, Homo sapiens
  CD4 transmembrane domain
PVQPMALIVLGGVAGLLLFIGLGIFFCVRCRHRRR

SEQ ID NO: 11 - DNA, 111 bp, Homo sapiens
  CD28 transmembrane domain
cctagcaagcctttttgggtgctcgtggtcgttggcggagtgctggcctgttatagcctgctggtcaccgtggccttcatcatcttttgggtccgaagcaagcggagcaga

SEQ ID NO: 12 - protein, 37 aa, Homo sapiens
  CD28 transmembrane domain
PSKPFWVLVVVGGVLACYSLLVTVAFIIFWVRSKRSR

SEQ ID NO: 13 - DNA, 96 bp, Homo sapiens
  CD55 GPI-anchor coding sequence
ggcactacttccggaaccaccagactgctgagcggtcacacgtgcttcaccctcacagggttgcttggcacactggtgaccatgggactcctgaca

SEQ ID NO: 14 - protein, 32 aa, Homo sapiens
  CD55 GPI-anchor coding sequence
GTTSGTTRLLSGHTCFTLTGLLGTLVTMGLLT

SEQ ID NO: 15 - DNA, 90 bp, Homo sapiens
  CD59 GPI-anchor coding sequence
cagctcgagaacggaggcacttctctgtccgaaaagaccgtgctgctgctggtcacacccttccttgcagctgcctggagcttgcaccct

SEQ ID NO: 16 - protein, 30 aa, Artificial Sequence
  CD59 GPI-anchor coding sequence
QLENGGTSLSEKTVLLLVTPFLAAAWSLHP

SEQ ID NO: 17 - DNA, 723 bp, Artificial Sequence
  mRuby3 fluorescent protein
gcctctggtaccgtgtccaagggcgaagaactgatcaaagaaaacatgcggatgaaggtggtcatggaaggcagcgtgaacggccaccagttcaagtgtacaggcgaaggcgagggcagaccttacgaaggcgtgcaagtcatgcggatcaaagtgatcgaaggcggccctctgccttttgccttcgatatcctggccaccagctttatgtacggcagccggaccttcatcaagtaccccgccgacattcccgatttcttcaagcagagcttccccgagggcttcacctgggagagagtgaccagatacgaggatggcggcgtcgtgaccgtgacacaggataccagtctggaagatggcgagctggtgtacaacgtgaaagtgcggggcgtgaacttccccagcaatggccctgtgatgcagaaaaagaccaaaggctgggagcccaacaccgagatgatgtaccctgctgatggcggcctgagaggctacacagatatcgccctgaaagtggacggcggaggccatctgcactgcaacttcgtgaccacctatcggtccaagaaaaccgtgggcaacatcaagatgcccggggtgcacgccgtggaccacagactggaaagaatcgaagagagcgacaacgagacatacgtggtgcagagagaagtggccgtcgccaagtacagcaatctcggaggcggaatggacgagctgtacaagtaa

SEQ ID NO: 18 - protein, 240 aa, Artificial Sequence
  mRuby3 fluorescent protein
ASGTVSKGEELIKENMRMKVVMEGSVNGHQFKCTGEGEGRPYEGVQVMRIKVIEGGPLPFAFDILATSFMYGSRTFIKYPADIPDFFKQSFPEGFTWERVTRYEDGGVVTVTQDTSLEDGELVYNVKVRGVNFPSNGPVMQKKTKGWEPNTEMMYPADGGLRGYTDIALKVDGGGHLHCNFVTTYRSKKTVGNIKMPGVHAVDHRLERIEESDNETYVVQREVAVAKYSNLGGGMDELYK

SEQ ID NO: 19 - DNA, 945 bp, Artificial Sequence
  miRFP720 fluorescent protein
gcggaggggagcgtagccaggcagcctgatttgctcacttgtgatgacgaaccgattcacatccctggagcaatacagcctcacgggctactgctcgcgttagcggctgacatgactattgttgcgggcagcgataacttaccagagctaacgggacttgcaatcggggctttgatagggcgatctgctgcagacgtgttcgattcggagacgcataaccgtttgaccatcgctttggctgagccgggggcagctgtcggagcccctattactgtcggctttactatgcgcaaggacgctggattcatcggctcttggcatcggcacgatcagctgatttttcttgaacttgaaccaccccagcgagatgtggccgaaccccaagcattctttcgcagaacaaactcagccatacgtcggctccaagcagcagagaccctcgagtcagcttgcgccgctgccgctcaggaagtccgaaaaatcacaggttttgatagggttatgatatatcggttcgccagtgattttagtggtgaagtcattgcagaggaccgctgtgccgaagtagagtctaaactgggactgcactaccctgcctcctttatccctgcccaggcccgcagactgtacaccattaatccagtccggataattccagacatcaattataggcccgttccagttacaccagaccttaatcccgtgacaggcagacccattgacctctccttcgccattctccggagcgtgagccccgtgcatctggaattcatgcgcaacatcggcatgcacggtaccatgtccatctccatcctgaggggtgagaggctgtggggcctgatcgtgtgccaccataggaccccctattacgtggacctggacggaagacaggcctgcaagagagtggccgagagactggccacccaaatcggcgtgatggaggag

SEQ ID NO: 20 - protein, 315 aa, Artificial Sequence
  miRFP720 fluorescent protein
AEGSVARQPDLLTCDDEPIHIPGAIQPHGLLLALAADMTIVAGSDNLPELTGLAIGALIGRSAADVFDSETHNRLTIALAEPGAAVGAPITVGFTMRKDAGFIGSWHRHDQLIFLELEPPQRDVAEPQAFFRRTNSAIRRLQAAETLESACAAAAQEVRKITGFDRVMIYRFASDFSGEVIAEDRCAEVESKLGLHYPASFIPAQARRLYTINPVRIIPDINYRPVPVTPDLNPVTGRPIDLSFAILRSVSPVHLEFMRNIGMHGTMSISILRGERLWGLIVCHHRTPYYVDLDGRQACKRVAERLATQIGVMEE

SEQ ID NO: 21 - DNA, 1482 bp, Artificial Sequence
  DTPA-V5-TMD-mRuby3
atgcctctcggactgctgtggcttggactggctctgctgggagcactgcatgctcaggcccaggatagcaccagcgatctgattccagctcctccactgagcaaggtgcccctgcagcagaacttccaggacaaccaattccatgggaagtggtatcaagtgggcagagccggaaatgccgctcctcctgaagatcctgaactgctgctgctgacagcccagacctacgagctgaaagaggacaagagctacgacgtgaccgccgtgcggttccggaagaagatgtgcgagtacctgaccatgaccttcgtgcctggatctcagcctggcgagttcaccctgggcaacatcaagaactaccccggcctgaccagctacttcgtcagagtggtgtccaccaactacaaccagcacgccatggtgttcttcaagaaggtccagcagaaccgcgagtacttcagcatctctctgctcggccggaccaaagagctgaccagcgaactgaaagagaacttcatccggttctccaaatctctgggcctgcccgagaatcacatcgtgttccccgtgcctatcgaccagtgcatcgatgccagcggcaagcccattcctaatcctctgctgggcctcgattctactggcgcgcctgcttctcctgttcaacccatggctctgatcgtgcttggcggagttgctggactgctgctgtttatcggcctgggcatcttcttttgcgtgcggtgcagacataggcggagagcctctggtaccgtgtccaagggcgaagaactgatcaaagaaaacatgcggatgaaggtggtcatggaaggcagcgtgaacggccaccagttcaagtgtacaggcgaaggcgagggcagaccttacgaaggcgtgcaagtcatgcggatcaaagtgatcgaaggcggccctctgccttttgccttcgatatcctggccaccagctttatgtacggcagccggaccttcatcaagtaccccgccgacattcccgatttcttcaagcagagcttccccgagggcttcacctgggagagagtgaccagatacgaggatggcggcgtcgtgaccgtgacacaggataccagtctggaagatggcgagctggtgtacaacgtgaaagtgcggggcgtgaacttccccagcaatggccctgtgatgcagaaaaagaccaaaggctgggagcccaacaccgagatgatgtaccctgctgatggcggcctgagaggctacacagatatcgccctgaaagtggacggcggaggccatctgcactgcaacttcgtgaccacctatcggtccaagaaaaccgtgggcaacatcaagatgcccggggtgcacgccgtggaccacagactggaaagaatcgaagagagcgacaacgagacatacgtggtgcagagagaagtggccgtcgccaagtacagcaatctcggaggcggaatggacgagctgtacaagtaa

SEQ ID NO: 22 - protein, 493 aa, Artificial Sequence
  DTPA-V5-TMD-mRuby3
MPLGLLWLGLALLGALHAQAQDSTSDLIPAPPLSKVPLQQNFQDNQFHGKWYQVGRAGNAAPPEDPELLLLTAQTYELKEDKSYDVTAVRFRKKMCEYLTMTFVPGSQPGEFTLGNIKNYPGLTSYFVRVVSTNYNQHAMVFFKKVQQNREYFSISLLGRTKELTSELKENFIRFSKSLGLPENHIVFPVPIDQCIDASGKPIPNPLLGLDSTGAPASPVQPMALIVLGGVAGLLLFIGLGIFFCVRCRHRRRASGTVSKGEELIKENMRMKVVMEGSVNGHQFKCTGEGEGRPYEGVQVMRIKVIEGGPLPFAFDILATSFMYGSRTFIKYPADIPDFFKQSFPEGFTWERVTRYEDGGVVTVTQDTSLEDGELVYNVKVRGVNFPSNGPVMQKKTKGWEPNTEMMYPADGGLRGYTDIALKVDGGGHLHCNFVTTYRSKKTVGNIKMPGVHAVDHRLERIEESDNETYVVQREVAVAKYSNLGGGMDELYK

SEQ ID NO: 23 - DNA, 774 bp, Artificial Sequence
  DTPA-V5-TMD
atgcctctcggactgctgtggcttggactggctctgctgggagcactgcatgctcaggcccaggatagcaccagcgatctgattccagctcctccactgagcaaggtgcccctgcagcagaacttccaggacaaccaattccatgggaagtggtatcaagtgggcagagccggaaatgccgctcctcctgaagatcctgaactgctgctgctgacagcccagacctacgagctgaaagaggacaagagctacgacgtgaccgccgtgcggttccggaagaagatgtgcgagtacctgaccatgaccttcgtgcctggatctcagcctggcgagttcaccctgggcaacatcaagaactaccccggcctgaccagctacttcgtcagagtggtgtccaccaactacaaccagcacgccatggtgttcttcaagaaggtccagcagaaccgcgagtacttcagcatctctctgctcggccggaccaaagagctgaccagcgaactgaaagagaacttcatccggttctccaaatctctgggcctgcccgagaatcacatcgtgttccccgtgcctatcgaccagtgcatcgatgccagcggcaagcccattcctaatcctctgctgggcctcgattctactggcgcgcctgcttctcctgttcaacccatggctctgatcgtgcttggcggagttgctggactgctgctgtttatcggcctgggcatcttcttttgcgtgcggtgcagacataggcggagagcctctaccggttaa

SEQ ID NO: 24 - protein, 257 aa, Artificial Sequence
  DTPA-V5-TMD
MPLGLLWLGLALLGALHAQAQDSTSDLIPAPPLSKVPLQQNFQDNQFHGKWYQVGRAGNAAPPEDPELLLLTAQTYELKEDKSYDVTAVRFRKKMCEYLTMTFVPGSQPGEFTLGNIKNYPGLTSYFVRVVSTNYNQHAMVFFKKVQQNREYFSISLLGRTKELTSELKENFIRFSKSLGLPENHIVFPVPIDQCIDASGKPIPNPLLGLDSTGAPASPVQPMALIVLGGVAGLLLFIGLGIFFCVRCRHRRRASTG

SEQ ID NO: 25 - DNA, 774 bp, Artificial Sequence
  Colchi-V5-TMD
atgcctctcggactgctgtggcttggactggctctgctgggagcactgcatgctcaggcccaggatagcaccagcgatctgattccagctcctccactgagcaaggtgcccctgcagcagaacttccaggacaaccaattccatggggagtggtatgtggtaggtgtcgccggcaatggtttcctcagagaagataaagatccgattaaaatggcggccaccatctacgaattaaaagaagacaagtcttataacgtgacctttatgaagtttccaatgaagaaatgcgagtacatgaccgacacgctggttcctggaagtcagcccggcgagtttatccttggcaatatcaagagtgaacctggatacacatcttggctcgtgcgcgtcgtgtcaacaaactataatcagcacgctatggtgttctttaaggctgttcaacagaatcgggaggactttttcattactctttacggaaggacaatagagctgacctccgagctgaaggagaactttattcgattctccaaatctctgggcctgcccgagaatcacatcgtgttccccgtgcctatcgaccagtgcatcgatgccagcggcaagcccattcctaatcctctgctgggcctcgattctactggcgcgcctgcttctcctgttcaacccatggctctgatcgtgcttggcggagttgctggactgctgctgtttatcggcctgggcatcttcttttgcgtgcggtgcagacataggcggagagcctctaccggttaa

SEQ ID NO: 26 - protein, 257 aa, Artificial Sequence
  Colchi-V5-TMD
MPLGLLWLGLALLGALHAQAQDSTSDLIPAPPLSKVPLQQNFQDNQFHGEWYVVGVAGNGFLREDKDPIKMAATIYELKEDKSYNVTFMKFPMKKCEYMTDTLVPGSQPGEFILGNIKSEPGYTSWLVRVVSTNYNQHAMVFFKAVQQNREDFFITLYGRTIELTSELKENFIRFSKSLGLPENHIVFPVPIDQCIDASGKPIPNPLLGLDSTGAPASPVQPMALIVLGGVAGLLLFIGLGIFFCVRCRHRRRASTG

SEQ ID NO: 27 - DNA, 1719 bp, Artificial Sequence
  DTPA-V5-TMD-miRFP720
atgcctctcggactgctgtggcttggactggctctgctgggagcactgcatgctcaggcccaggatagcaccagcgatctgattccagctcctccactgagcaaggtgcccctgcagcagaacttccaggacaaccaattccatgggaagtggtatcaagtgggcagagccggaaatgccgctcctcctgaagatcctgaactgctgctgctgacagcccagacctacgagctgaaagaggacaagagctacgacgtgaccgccgtgcggttccggaagaagatgtgcgagtacctgaccatgaccttcgtgcctggatctcagcctggcgagttcaccctgggcaacatcaagaactaccccggcctgaccagctacttcgtcagagtggtgtccaccaactacaaccagcacgccatggtgttcttcaagaaggtccagcagaaccgcgagtacttcagcatctctctgctcggccggaccaaagagctgaccagcgaactgaaagagaacttcatccggttctccaaatctctgggcctgcccgagaatcacatcgtgttccccgtgcctatcgaccagtgcatcgatgccagcggcaagcccattcctaatcctctgctgggcctcgattctactggcgcgcctgcttctcctgttcaacccatggctctgatcgtgcttggcggagttgctggactgctgctgtttatcggcctgggcatcttcttttgcgtgcggtgcagacataggcggagagcctctaccggtgcggaggggagcgtagccaggcagcctgatttgctcacttgtgatgacgaaccgattcacatccctggagcaatacagcctcacgggctactgctcgcgttagcggctgacatgactattgttgcgggcagcgataacttaccagagctaacgggacttgcaatcggggctttgatagggcgatctgctgcagacgtgttcgattcggagacgcataaccgtttgaccatcgctttggctgagccgggggcagctgtcggagcccctattactgtcggctttactatgcgcaaggacgctggattcatcggctcttggcatcggcacgatcagctgatttttcttgaacttgaaccaccccagcgagatgtggccgaaccccaagcattctttcgcagaacaaactcagccatacgtcggctccaagcagcagagaccctcgagtcagcttgcgccgctgccgctcaggaagtccgaaaaatcacaggttttgatagggttatgatatatcggttcgccagtgattttagtggtgaagtcattgcagaggaccgctgtgccgaagtagagtctaaactgggactgcactaccctgcctcctttatccctgcccaggcccgcagactgtacaccattaatccagtccggataattccagacatcaattataggcccgttccagttacaccagaccttaatcccgtgacaggcagacccattgacctctccttcgccattctccggagcgtgagccccgtgcatctggaattcatgcgcaacatcggcatgcacggtaccatgtccatctccatcctgaggggtgagaggctgtggggcctgatcgtgtgccaccataggaccccctattacgtggacctggacggaagacaggcctgcaagagagtggccgagagactggccacccaaatcggcgtgatggaggagtaa

SEQ ID NO: 28 - protein, 572 aa, Artificial Sequence
  DTPA-V5-TMD-miRFP720
MPLGLLWLGLALLGALHAQAQDSTSDLIPAPPLSKVPLQQNFQDNQFHGKWYQVGRAGNAAPPEDPELLLLTAQTYELKEDKSYDVTAVRFRKKMCEYLTMTFVPGSQPGEFTLGNIKNYPGLTSYFVRVVSTNYNQHAMVFFKKVQQNREYFSISLLGRTKELTSELKENFIRFSKSLGLPENHIVFPVPIDQCIDASGKPIPNPLLGLDSTGAPASPVQPMALIVLGGVAGLLLFIGLGIFFCVRCRHRRRASTGAEGSVARQPDLLTCDDEPIHIPGAIQPHGLLLALAADMTIVAGSDNLPELTGLAIGALIGRSAADVFDSETHNRLTIALAEPGAAVGAPITVGFTMRKDAGFIGSWHRHDQLIFLELEPPQRDVAEPQAFFRRTNSAIRRLQAAETLESACAAAAQEVRKITGFDRVMIYRFASDFSGEVIAEDRCAEVESKLGLHYPASFIPAQARRLYTINPVRIIPDINYRPVPVTPDLNPVTGRPIDLSFAILRSVSPVHLEFMRNIGMHGTMSISILRGERLWGLIVCHHRTPYYVDLDGRQACKRVAERLATQIGVMEE

SEQ ID NO: 29 - protein, 6 aa, Homo sapiens
  Amyloid beta sequence recognized by an Anticalin
VFFAED